(19) United States
(12) Patent Application Publication
(10) Pub. No.: US 2010/0280366 A1
(43) Pub. Date: Nov. 4, 2010

(54) CONTINUOUS FIELD TOMOGRAPHY SYSTEMS AND METHODS OF USING THE SAME

(76) Inventors: Lawrence Arne, Redwood City, CA (US); Orlando Banos, Sunnyvale, CA (US); Yashar Behzadi, San Francisco, CA (US); Olivier Colliou, Sunnyvale, CA (US); Benedict Costello, Berkeley, CA (US); Robert Leichner, Menlo Park, CA (US); Gregory Moon, Orinda, CA (US); Timothy Robertson, Belmont, CA (US); George Savage, Portola Valley, CA (US); Todd Thompson, San Jose, CA (US); Mark Zdeblick, Portola Valley, CA (US)

Correspondence Address: Proteus Biomedical, Inc.;Bozicevic, Field & Francis LLP, 1900 University Avenue, Suite 200, East Palo Alto, CA, 94303, US

(21) Appl. No.: 12/522,537

(22) PCT Filed: May 12, 2009
(86) PCT No.: PCT/US09/43653

§ 371 Date: Jul. 8, 2009

Related U.S. Application Data
(60) Provisional application No. 61/052,952, filed on May 13, 2008.
(60) Provisional application No. 61/075,671, filed on Jun. 25, 2008.
(60) Provisional application No. 61/075,673, filed on Jun. 25, 2008.
(60) Provisional application No. 61/075,670, filed on Jun. 25, 2008.
(60) Provisional application No. 61/076,577, filed on Jun. 27, 2008.

(51) Int. Cl. A61B 5/05

(52) U.S. Cl. 600/425

(57) ABSTRACT

Continuous field tomography systems are provided. Aspects of systems include a data aggregating module configured to receive both continuous field tomography data and non-continuous field physiological data and produce an aggregated data product from these disparate types of data. Also provided are methods of using systems of the invention in a variety of different applications, including diagnostic and therapeutic applications. The systems and methods of the invention find use in a variety of different applications, such as cardiac related applications.

### CROSS-REFERENCE TO RELATED APPLICATIONS

Pursuant to 35 U.S.C. §119(e), this application claims priority to the filing date of U.S. Provisional Patent Application Ser. No. 61/053,952 filed May 13, 2008; U.S. Provisional Patent Application Ser. No. 61/075,671 filed Jun. 25, 2008; U.S. Provisional Patent Application Ser. No. 61/075,673 filed Jun. 25, 2008; U.S. Provisional Patent Application Ser. No. 61/075,670 filed Jun. 25, 2008; and U.S. Provisional Patent Application Ser. No. 61/076,577 filed Jun. 27, 2008; the disclosures of which applications are herein incorporated by reference.

### INTRODUCTION

Continuous field tomography is an important new tool for evaluating movement of tissue in a subject. One type of continuous field tomography is electric tomography. Electric tomography (ET) generally refers to imaging through use of an applied electric field. In an electric tomography system, an electrical field generator may generate the electrical field which is applied to a subject, e.g., a patient. In the ET system, a sensor electrode may be stably associated with a tissue site, e.g., an electrical lead having the sensor electrode physically associated with an organ. The sensor electrode then generates an induced signal in response to the electrical field applied to it. The induced signal, which corresponds to displacement of the sensor electrode or the tissue site, is forwarded to a signal processing module which processes the induced signal for various applications. By processing the induced signal, the displacement, velocity, and/or other data associated with the sensor electrode or the movement of the tissue site may be obtained.

### SUMMARY

Continuous field tomography systems are provided. Aspects of systems include a data aggregating module configured to receive both continuous field tomography data and non-continuous field physiological data and produce an aggregated data product from these disparate types of data. Also provided are methods of using systems of the invention in a variety of different applications, including diagnostic and therapeutic applications. The systems and methods of the invention find use in a variety of different applications, such as cardiac related applications.

### BRIEF DESCRIPTION OF THE FIGURES

FIG. 1 is an illustration of an ET sensing capable ICD in association with a patient's heart, according to one embodiment of the invention

FIG. 2 shows a functional block diagram illustrating the steps employed in an embodiment of the present invention for verifying an ECG-based diagnosis using an ET signal.

FIG. 3 shows a second functional block diagram illustrating the steps employed in an embodiment of the present invention for incorporating baseline statistical data in diagnosis.

FIG. 4 shows a third functional block diagram illustrating the steps employed in an embodiment of the present invention for adjusting ECG signal sensitivity using ET signal data.

FIG. 5 provides a view of a system that simultaneously evaluates pacing performance and measures cardiac performance using continuous field tomography.

FIG. 6 provides a view of a system such as shown in FIG. 5, but where the pacing system analyzer function is separated from the electrical tomography function.

FIG. 7 provides a view of a system analogous to that shown in FIG. 5, but where the system further includes a programmer for an implantable pacemaker.

FIG. 8 provides a view of a system analogous to that shown in FIG. 7, but where wired connections have been replaced with wireless connections.

FIG. 9 provides a view of a system that simultaneously measures cardiac performance using continuous field tomography and provides a physician or other healthcare professional a way of monitoring the cardiac performance.

FIG. 10 provides a view of a system analogous to that shown in FIG. 8, with the exception that the console of FIG. 8 has been replaced with a Holter recorder.

FIG. 11 provides a view of a system that includes a loop recorder.

FIG. 12 provides a view of a system in which the implantable pulse generator is a right atrium only pacemaker.

FIG. 13 provides a view of a system in which the pulse generator is connected to a right ventricular lead to provide right ventricular pacing and also measure cardiac performance derived from the motion of the right ventricular lead.

FIG. 14 illustrates a cardiac resynchronization therapy pacemaker that has three leads.

FIG. 15 depicts a system where a CRT pace generator is connected to a programmable multi-electrode lead in the left ventricle.

FIG. 16 depicts a system where an electrical tomography cardiac resynchronization pacemaker includes an epicardial lead.

FIG. 17 illustrates a first sample user display, according to an embodiment of the present invention.

FIG. 18 illustrates a second sample user display, according to an embodiment of the present invention.

FIG. 19 illustrates a third sample user display, according to an embodiment of the present invention.

FIG. 20 illustrates a fourth sample user display, according to an embodiment of the present invention.

FIG. 21 provides a view of an image that is an aggregated product of both CT and ET data.

FIG. 22 provides a view of an image that is an aggregated product of both CT/PET and ET data.

FIG. 23 provides a view of an image that is an aggregated product of CT data, ET data and ECG data.

### DETAILED DESCRIPTION

Continuous field tomography systems are provided. Aspects of systems include a data aggregating module configured to receive both continuous field tomography data and non-continuous field physiological data and produce an aggregated data product from these disparate types of data. Also provided are methods of using systems of the invention in a variety of different applications, including diagnostic and therapeutic applications. The systems and methods of the invention find use in a variety of different applications, such as cardiac related applications.

As summarized above, systems of the invention include a processor configured to receive both continuous field tomography data and non-continuous field physiological data and produce an aggregated data product from these disparate types of data. As such, systems of the invention are structured to receive at least two disparate types of data. One of these types of data is continuous field tomography data. Another of these types of data is non-continuous field physiological data. Each of these types of data is obtained from a living subject of interest, .e.g., a patient that is being diagnosed and/or treated for disease condition or conditions.

Continuous field tomography data is data obtained from a living subject by using a continuous field tomography method, where the method may employ a continuous field tomography data source, e.g., a device configured to provide continuous field tomography data. By “continuous field tomography method” is meant a method which employs detected changes in an applied continuous field to obtain a signal, which signal is then employed to determine movement of a body associated object of interest, such as a tissue location or an implanted device. For the purposes of this application, the term “continuous field” means a field from which tomography measurement data is obtained from the field's continuous aspect.

Where desired, object movement can be determined relative to a reference, such as a second tissue location or object, such that the methods are employed to determine movement of two or more objects relative to each other.

As mentioned above, the object can be a tissue location or another body-associated structure, such as an implanted device. Where the object is a tissue location, the tissue location is a defined location or portion of a body, such as an organ. In some instances, the tissue location is an internal body structure, such as an internal organ, e.g., heart, kidney, stomach, lung, etc. Of interest in certain embodiments are cardiac locations, where the cardiac location may be either endocardial or epicardial, as desired, and may be an atrial or ventricular location.

To obtain continuous field data, a continuous field is produced in a manner such that the object of interest, such as the tissue location or implanted device, is present in the generated continuous field. In certain embodiments, a single continuous field is generated, while in other embodiments a plurality of different continuous fields are generated, e.g., two or more, such as three or more, where in certain of these embodiments, the generated continuous fields may be substantially orthogonal to one another. Of interest in some embodiments is the production of three different orthogonal continuous fields, where there is a continuous field for each of the X, Y and Z axes.

In practicing the subject methods, the applied continuous field may be applied using any convenient format, e.g., from outside the body, from an internal body site, or a combination thereof, so long as the object of interest resides in the applied continuous field. As such, in certain embodiments the applied continuous field is applied from an external body location, e.g., from a body surface location. In yet other embodiments, the continuous field is generated from an internal site, e.g., from an implanted device.

Following generation of the applied continuous field, as described above, a signal (representing data) from a continuous field sensing element that is stably associated with the object of interest is then detected to evaluate movement of the object. A signal from the sensing element may be detected at least twice over a given period of time, for example to determine whether a parameter(s) being sensed by the sensing element has changed or not over the period of time, and therefore whether or not the object of interest has moved over the period of time of interest. In certain embodiments, a change in a parameter is detected by the sensing element to evaluate movement of the object.

In obtaining continuous field tomography data, at least one parameter of the applied continuous field may be detected by the sensing element at two or more different times. Parameters of interest include, but are not limited to: amplitude, phase and frequency of the applied continuous field, as reviewed in greater detail below. In certain embodiments, the parameter of interest is detected at the two or more different times in a manner such that one or more of the other of the three parameters is substantially constant, if not constant.

By “stably associated with” is meant that the sensing element is substantially if not completely fixed relative to the object of interest such that when the object of interest moves, the sensing element also moves. As the employed continuous field sensing element is stably associated with the object of interest, movement of the sensing element is at least a proxy for, and in certain embodiments is the same as, the movement of the object of interest to which it is stably associated, such that movement of the sensing element can be used to evaluate movement of the object of interest. The continuous field sensing element may be stably associated with the object of interest using any convenient approach, such as by attaching the sensing element to the tissue location by using an attachment element, such as a hook, etc., by having the sensing element on a structure that compresses the sensing element against the tissue location such that the two are stably associated, by fixing the sensing element on an implanted device which is the object of interest, etc.

In obtaining continuous field data, a single sensing element may be employed. In such instances, evaluation may include monitoring movement of the object over a given period of time. In certain embodiments, two or more distinct sensing elements are employed to evaluate movement of two or more objects of interest, such as two or more tissue locations, two or more locations of implanted device, etc. The number of different sensing elements that are employed in a given embodiment may vary greatly, where in certain embodiments the number employed is 2 or more, such as 3 or more, 4 or more, 5 or more, 8 or more, 10 or more, etc. In such multi-sensor embodiments, the methods may include evaluating movement of the two or more distinct object locations relative to each other.

The nature of the applied continuous field employed may vary. In some embodiments, the continuous field that is applied is a wave field. In some embodiments, the wave field is an electromagnetic wave. Electromagnetic continuous fields of interest include electrical and magnetic fields, as well as light. In yet other representative embodiments, the wave field is a pressure wave, where a representative continuous field of this type is an acoustic field.

In some instances, the continuous field tomography data is electric tomography data. Electric tomography data is data obtained by an electric field tomography method. By “electric field tomography method” is meant a method which employs detected changes in an applied electric field to obtain a signal, which signal is then employed to determine tissue location movement. For the purposes of this application, the term “electric field” means an electric field from which tomography measurement data is obtained. The electric field is one or more cycles of a sine wave. There is no necessary requirement for discontinuity in the field to obtain data. As such, the applied field employed in embodiments of the subject invention is continuous over a given period of time. Electric field tomography data can be based upon measurement of the amplitude, frequency, and phase shift of the induced signal.

In obtaining electric field tomography data, the applied electric field(s) may be applied using any convenient format, e.g., from outside the body, from an internal body site, or a combination thereof, as long as the object of interest resides in the applied electric field. The employed electric field or fields may be produced using any convenient electric field generation element, where in certain embodiments the electric field is set up between a driving electrode and a ground element, e.g., a second electrode, an implanted medical device that can serve as a ground, such as a “can” of an implantable cardiac device (for example a pacemaker), etc. The electric field generation elements may be implantable such that they generate the electric field from within the body, or the elements may be ones that generate the electric field from locations outside of the body, or a combination thereof. As such, in certain embodiments the applied electric field is applied from an external body location, e.g., from a body surface location. In yet other embodiments, the electric field is generated from an internal site, e.g., from an implanted device (such as a pacemaker can), one or more electrodes on a lead, such as a multiplexed electric lead. Multiplex leads that find use in these latter embodiments include, but are not limited, those described in those described in U.S. Pat. No. 7,214,189 and U.S. patent application Ser. No. 10/734,490 published as 20040193021; the disclosures of which are herein incorporated by reference.

In certain embodiments, the electric field is a radiofrequency or RF field. In these instances, the electric field generation element generates an alternating current electric field, e.g., that comprises an RF field, where the RF field has a frequency ranging from about 1 kHz to about 100 GHz or more, such as from about 10 kHz to about 10 MHz, including from about 25 KHz to about 1 MHz. Aspects of this embodiment of the present invention involve the application of alternating current within the body transmitted between two electrodes with an additional electrode pair being used to record changes in a property, e.g., amplitude, within the applied RF field. Several different frequencies can be used to establish different axes and improve resolution, e.g., by employing either RF energy transmitted from a subcutaneous or cutaneous location, in various planes, or by electrodes, deployed for example on an inter-cardiac lead, which may be simultaneously used for pacing and sensing. Where different frequencies are employed simultaneously, the magnitude of the difference in frequencies will, in certain embodiments, range from about 100 Hz to about 100 KHz, such as from about 5 KHz to about 50 KHz. Amplitude information can be used to derive the position of various sensors relative to the emitters of the alternating current.

Following generation of the applied electric field, a signal (representing data) from an electric field sensing element that is stably associated with the object of interest is then detected to evaluate movement of the object of interest. A signal from the sensing element may be detected at least twice over a given duration of time to determine whether a parameter(s) being sensed by the sensing element has changed or not over the period of time, and therefore whether or not the object of interest has moved over the period of time of interest. Parameters of interest include, but are not limited to: amplitude, phase and frequency of the applied electric field, as reviewed in greater detail below. In certain embodiments, the parameter of interest is detected at the two or more different times in a manner such that one or more of the other of the three parameters is substantially constant, if not constant. In a given embodiment, the sensing element can provide output in an interval fashion or continuous fashion for a given duration of time, as desired.

The sensing element is, in certain embodiments, an electric potential sensing element, such as an electrode. In these embodiments, the sensing element provides a value for a sensed electric potential which is a function of the location of the sensing element in the generated electric field. In certain embodiments, the electric field sensing element is an electrode. The electrode may be present as a stand alone device, e.g., a small device that wirelessly communicates with a data receiver, or part of a component device, e.g., a medical carrier, such as a lead. Where the sensing element is an electrode on a lead, the lead may be a conventional lead that includes a single electrode. In alternative embodiments, the lead may be a multi-electrode lead that includes two or more different electrodes, where in certain of these embodiments, the lead may be a multiplex lead that has two or more individually addressable electrodes electrically coupled to the same wire or wires. In certain embodiments, a lead, such as a cardiovascular lead, is employed that includes one or more sets of electrode satellites (for example electrode satellites that are electrically coupled to at least one elongated conductive member, e.g., an elongated conductive member present in the lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex multi-electrode lead structures of interest include those described in U.S. Pat. No. 7,214,189 and U.S. patent application Ser. No. 10/734,490 published as 20040193021; the disclosures of which are herein incorporated by reference.

In certain embodiments, the multiplex lead includes satellite electrodes that are segmented electrodes, in which two or more different individually addressable electrodes are coupled to the same satellite controller, e.g., integrated circuit, present on the lead. Segmented electrode structures of interest include, but are not limited to, those described in U.S. Pat. No. 7,214,189; PCT Application Serial No. PCT/US2005/46811 published as WO 2006/069322; and PCT Application Serial No PCT/US2005/46815 published as WO 2006/069323; the disclosures of the various segmented multiplex lead structures of these applications being herein incorporated by reference.

Continuous field tomography, including electric tomography, systems and methods that may be employed to obtain the continuous field tomography data employed in systems of the invention include those systems and methods further described in U.S. application Ser. Nos. 11/664,340; 11/731,786; 11/562,690; 12/037,851; 11/219,305; 11/793,904; 12/171,978; 11/909,786; the disclosures of which are herein incorporated by reference.

In addition to continuous field tomography data, systems of the invention are configured to receive non-continuous field physiological data, which is a type of data that is disparate from the continuous field tomography data as reviewed above. This non-continuous field physiological data may vary widely, so long as it is distinct from continuous field tomography data and is obtained from a non-continuous field physiological data source.

Non-continuous field physiological data sources of interest may vary depending on a particular application. One type of non-continuous field physiological data source is an implanted device, such as an implanted effector. Effectors of interest include both sensors and actuators. Sensors may comprise any suitable sensors such as pressure sensors, volume sensors, dimension sensors, temperature or thermal sensors, oxygen or carbon dioxide sensors, electrical conductivity sensors, electrical potential sensors, pH sensors, chemical sensors, flow rate sensors, optical sensors, acoustic sensors, hematocrit sensors, viscosity sensors and the like. An actuator may perform any suitable function, such as providing an electrical current or voltage, setting an electrical potential, generating a biopotential, pacing a heart, heating a substance or area, inducing a pressure change, releasing or capturing a material, emitting light, emitting sonic or ultrasound energy, emitting radiation, delivery an active agent to a site, or the like.

The implanted effector may be configured in a variety of different ways. Examples of effectors include implanted medical devices, such as implanted electrical stimulation devices, implanted data recorders, implanted drug delivery devices, etc. Of interest in certain embodiments are implanted cardiac devices, such as implantable cardioverter defibrillators (ICDs); pacemakers, and the like. Where desired, the implantable effector may be a multiplexed multi-electrode lead that contains effectors for sampling continuous field tomography data, providing electrical stimulation, and receiving data from an additional sensor, or some combination of the above. As an example, a left ventricle pacing lead may be constructed with distal multiplexed electrodes for stimulating the left ventricle freewall, additional multiplexed electrodes more proximal for measuring the motion of the mitral valve using electrical tomography, and a multiplexed pressure sensor for monitoring the right atrial pressure. These three sets of effectors may share the same one- or two-wire power and communication bus that traverses the length of the lead. The different communication packets may be distinguished from one another by time-division multiplexing, frequency-division multiplexing, code-division multiplexing or the like.

In some instances, the non-continuous field physiological data source is an extra-corporeal device. By extra-corporeal devices is meant a device that, when it obtains physiological data, at least a portion of it exists outside of the body, such that it is not an implanted device. The device may be a body-associated device, such that is contacts a topical surface of the body, or a device that is not in contact with the body. Body-associated devices of interest include, but are not limited to: body-associated signal receivers. Body-associated signal receivers of interest include, but are not limited to, conductively transmitted signal receivers, such as those receivers described in: PCT Application Serial No. PCT/US08/85048; PCT Application Serial No. PCT/US2007/024225 published as WO 2008/095183; PCT Application Serial No. PCT/US2007/024225 published as WO 2008/063626 and PCT Application Serial No. US2006/016370 published as WO 2006/116718; as well as U.S. Provisional Application Ser. No. 61/160,289; the disclosures of which are herein incorporated by reference.

Extra-corporeal devices of interest further include extracorporeal diagnostic devices, such as extra-corporeal physiological parameter measuring devices, imaging devices, and the like. Examples of such extra-corporeal devices include, but are not limited to: cardiac scintigraphy devices, echocardiography devices (including stress devices (such as exercise treadmill based devices) and bedside or ambulatory monitors); fluoroscopy devices; computed tomography devices; cardiovascular magnetic resonance devices; pulmonary artery catheter devices; etc.

Also of interest as sources of non-continuous field physiological data are ingestible event markers. Ingestible event markers are ingestible compositions that, upon contact with a target physiological site (such as the stomach) emit a detectable signal. Ingestible event marker systems include ingestible event markers and a receiver configured to receive a signal emitted by the ingestible event marker. Ingestible event markers of interest and systems thereof include, but are not limited to, those described in PCT application serial no. PCT/US2006/016370 published as WO/2006/116718; PCT application serial no. PCT/US2007/082563 published as WO/2008/052136; PCT application serial no. PCT/US2007/024225 published as WO/2008/063626; PCT application serial no. PCT/US2007/022257 published as WO/2008/066617; PCT application serial no. PCT/US2008/052845 published as WO/2008/095183; PCT application serial no. PCT/US2008/053999 published as WO/2008/101107; PCT application serial no. PCT/US2008/056296 published as WO/2008/112577; PCT application serial no. PCT/US2008/056299 published as WO/2008/112578; and PCT application serial no. PCT/US2008/077753 published as WO 2009/042812; the disclosures of which applications are herein incorporated by reference.

As reviewed above, the data aggregating modules of the invention are configured to receive continuous field tomography data and non-continuous field physiological data and then produce an aggregated data product from these two disparate types of data. In various aspects, data may be aggregated by the data aggregating module to produce the aggregated data product in a variety of ways. For example, multiple data streams from various sources may be combined, various types of data may be combined, various types of data may be processed, etc., in producing the aggregated data product. In aggregating the data, data from disparate streams may be maintained as separate but combined data sources, or the data from disparate streams may be processed in some manner to provide new data, which new data is the product of some manipulation of the data from the disparate sources.

The aggregated data product may vary widely. In some instances, the aggregated data product comprises information that is configured to be employed by a user, such as a health care professional, in some manner. The aggregated data product of such embodiments may be provided to a user by any convenient communication protocol. For example, the system may provide simple signals, such as audio or visual alert signals, to a user. Alternatively, the aggregated data product may be displayed to a user, for example by a graphical user interface presented to a user by an image display unit. In these instances, the user may use the aggregated data product in a variety of different ways, such as by modifying one or more operational parameters of a medical device, by making diagnostic decisions regarding whether a patient has a condition of interest, etc. In some instances, the aggregated data product may include information that is employed by a device to automatically modify an operating parameter of the device. For example, the aggregated data product may be provided to an implantable medical device, where upon receipt of the aggregated data product, the implantable medical device may be configured to change an operating parameter in some manner, for example activate an actuator, etc.

In various aspects, the above described data aggregating module may be implemented as software, e.g., digital signal processing software; hardware, e.g., a circuit; or combinations thereof.

In addition to the data aggregating module, systems of the invention may include a number of additional components. For example, systems of the invention may include sources of continuous field tomography data and sources of non-continuous field physiological data. Systems of the invention may further include communications modules, which may operate by wired or wireless protocols. The above additional components that may be present are merely examples, and not provided in a manner that limits the scope of the systems claimed herein.

The above provides a description of various aspects of systems that include a data aggregating module according to the invention. In further describing various aspects of the invention, the following sections provide details about specific embodiments of the systems and methods for their use.

### Electrical Tomography Enhanced Arrhythmia Detection

Electrical tomography (ET) enhanced arrhythmia detection comprises comparison of electrocardiography (ECG) data with ET obtained myocardial motion data as detected by ET sensors to improve the accuracy of diagnoses based on ECG data. Specifically, in this embodiment of the invention, ET data is used to verify an implantable cardioverter defibrillator (ICD) system's determination that administration of therapy is necessary. This embodiment is an example of a system in which the aggregated data product is a combination of ET and ECG data, where the aggregated data product is used by the ICD to modify an operating parameter of the ICD, i.e., to make a determination of whether or not to provide electrical stimulation to the subject.

ET as applied to tissue motion detection involves generating an electric field in the body and using an electric field sensing element stably associated with tissue to detect changes in the electric field as the tissue moves. The electric field may be produced, for example, between a driving electrode and a ground element such as the can of an ICD. The sensing element may be fixed relative to a cardiac location, for instance, a heart wall. Heart wall motion may thus be translated into an electrical signal representing the position of the sensor relative to a localized electric field.

Various implantable medical devices rely on electrical signals representing body function to determine when treatment is necessary. For example, ICDs sense electrical activity representative of heart motion to determine when shock treatment is to be administered. However, misinterpretation of signals may arise due to conditions such as oversensitivity, interference, or lead damage. Thus, verification of information derived from these signals is desirable. In the present invention, ET data is used to verify information derived from one or more electrical signals collected by an implantable medical device.

For example, ET data may be used to verify the accuracy of ECG signals collected by an ICD system. In one embodiment, when the ICD diagnoses an arrhythmia based on interpretation of ECG data, the ICD queries ET data to determine that motion characteristic of the arrhythmia has also occurred. If the ET data confirms the diagnosis, treatment is administered. However, if the diagnosis is not confirmed, treatment may be withheld, or further analysis may take place to determine whether treatment is appropriate.

One advantage of using ET signals to verify ECG signals is that both types of signal can be sensed using the same lead, such as a multiplexed multi-electrode lead as described above. Thus, an embodiment of the invention incorporates one or more leads capable of both ECG and ET sensing. A further advantage is that ET signal detection does not require mechanical or other elements that may be subject to malfunction and/or degradation issues not arising in electrical sensors.

Using ET to measure heart wall motion provides an alternative signal that can be utilized to verify information derived from the ECG signal. In this manner, ECG signal interpretation errors may be reduced or eliminated. In certain embodiments, the present invention eliminates or significantly reduces the rate of improper shock from an ICD.

ET signal information may be provided to the medical device by various means. For example, one or more ET sensors may be attached to an electrode lead. The number of ET sensor electrodes on a lead may vary, ranging in some instances from one to ten, such as three to seven, including five sensor electrodes on a lead. Likewise, the number of ECG signal sensor electrodes on a lead may vary, ranging in some instances from one to ten, such as three to seven, including five sensor electrodes on a lead. Various embodiments of the invention may utilize one to ten multi-electrode electrode leads, such as two to five multi-electrode leads, including 3 multi-electrode leads. In some embodiments, a lead will have both an ET sensor and an ECG sensor. Other embodiments will have multiple ET sensors and/or ECG sensors.

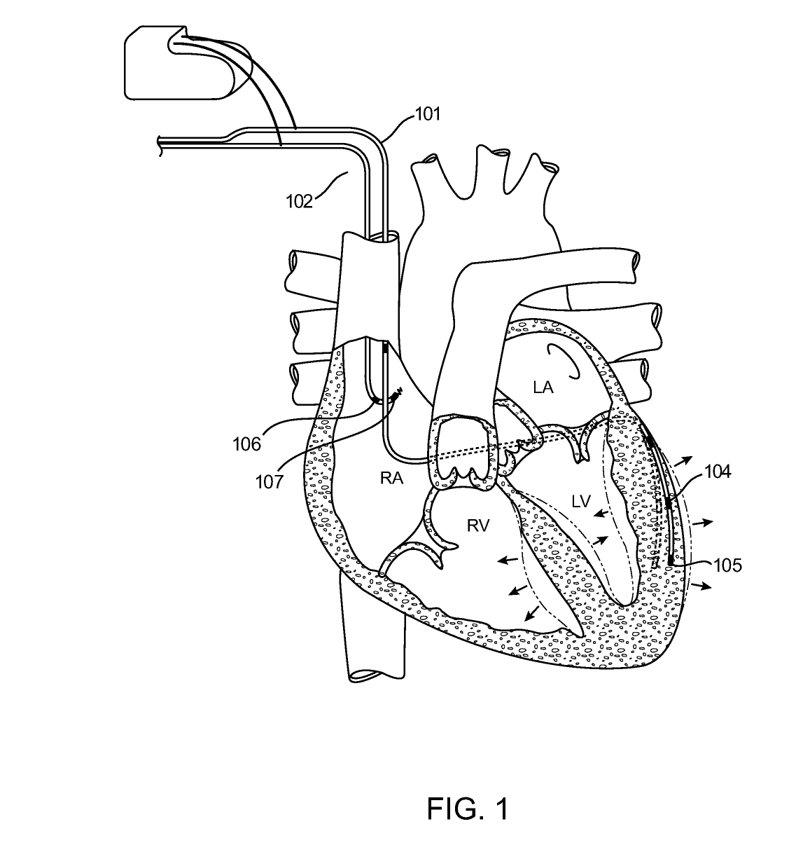

FIG. 1 provides a cross-sectional view of the heart with a two-lead embodiment of an ICD control system incorporating ET sensing technology. The system includes an ICD, a right atrium electrode lead 102 and left ventricle electrode lead 101. The left ventricle electrode lead 101 is shown equipped with an electrode 104 sensitive to cardiac electrical activity and electrode 105 for detection of an ET signal. The right atrium electrode lead 102 is shown equipped with an electrode 106 sensitive to cardiac electrical activity and electrode 107 for detection of an ET signal.

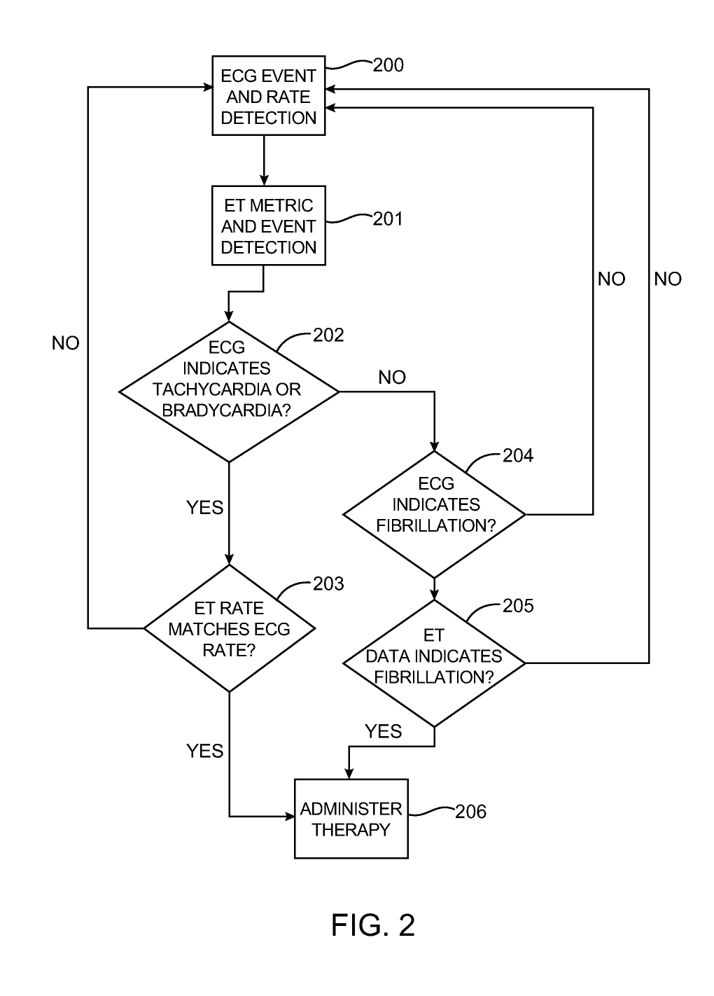

In one embodiment, the ICD control system compares the ECG signal with the ET signal representing heart wall motion to determine whether ECG data accurately represents heart activity. The flow chart in FIG. 2 presents an example of such an embodiment. ECG and ET detection, steps 200 and 201 respectively, are ongoing during the operation of the ICD. The ICD analyzes ECG and ET input data to determine various metrics, e.g., rate information such as R-to-R interval, and event information, e.g. occurrence of chaotic motion characteristic of defibrillation. When the ICD analysis of the ECG signal indicates an arrhythmia event, for example ventricular tachycardia, fast ventricular tachycardia, atrial tachycardia, ventricular fibrillation, atrial fibrillation, or bradycardia, the ET signal is queried to verify that the event has occurred. In the case that ECG input indicates tachycardia or bradycardia, the ICD system will determine whether the information provided by the ET signal confirms this diagnosis (steps 202-203). When the ECG signal indicates fibrillation, the ICD examines the ET signal for signs of fibrillation (steps 204-205). Only when ET data confirms the ECG diagnosis is therapy administered (step 206). In other embodiments, this analysis is augmented by additional criteria resulting in a range of algorithms with varying degrees of sophistication.

Data from one or more ET sensors may be used by the implantable medical device. Each ET sensor may be substantially or completely fixed relative to a defined location of the body. Generally this location will be the heart where the implantable medical device needing signal verification is an ICD. The cardiac location may be endocardial or epicardial and may be an atrial or ventricular location. In certain embodiments, the location will be a heart wall. The ICD control algorithm may incorporate one or more ET signal metrics, such as displacement, velocity, acceleration, and/or vector of movement. In one embodiment, the ICD collects data indicating relative motion between multiple ET sensors installed at different cardiac locations. For instance, the ICD algorithm may take a continuous net vector of motion from different points in the heart as an indication of fibrillation. As another example, one or more sensors may be used to determine whether, in the left side of the heart, the mitral valve is moving toward the apex. Absence of this motion could be taken as an indication of fibrillation. Thus, in one embodiment, when the ICD control algorithm determines from the ECG signal that a fibrillation event is occurring, the algorithm would proceed to evaluate ET signal data to determine whether it too indicated a fibrillation event was occurring (see FIG. 2, steps 204-205, supra).

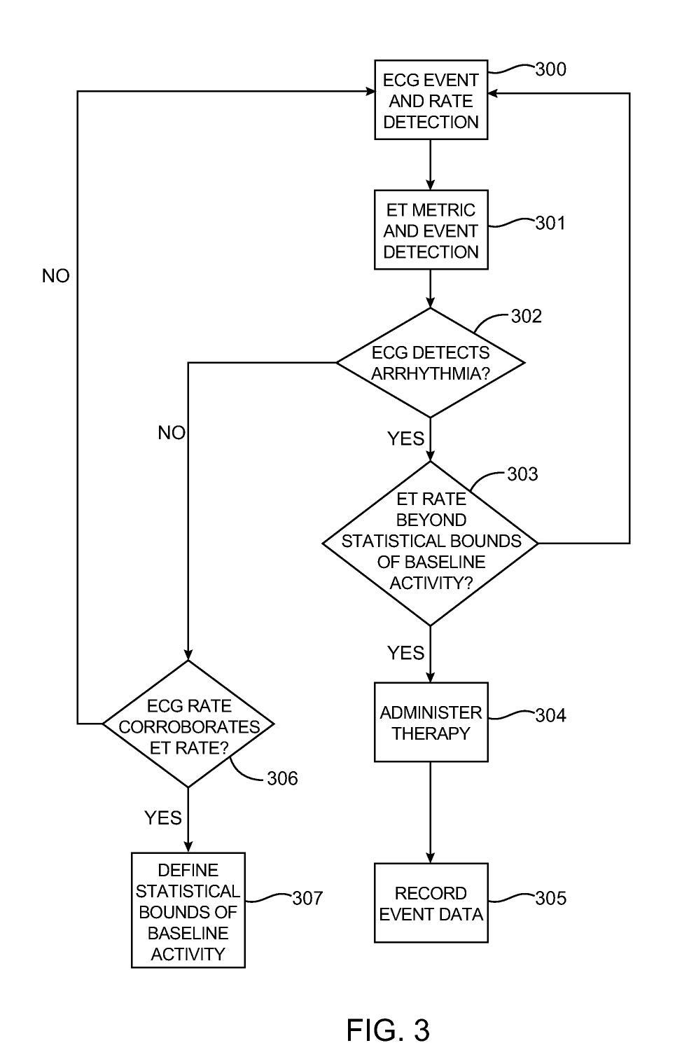

In some embodiments of the invention, the ICD system contains a memory element capable of storing historical ET sensor data. Historical data may used to create statistical bounds reflecting normal heart function. Metrics used for this purpose may include, for example, signal amplitude, pulse rate, and duration of event. Deviation beyond statistical bounds, or lack of such deviation, may be used to indicate whether an arrhythmia event is occurring. FIG. 3 is a flow chart demonstrating one possible application of this feature. When the ECG input indicates arrhythmia or a fibrillation event (step 302), the ICD control algorithm would determine whether the ET input exceeds/falls below one or more thresholds based on historical statistical bounds of normal heart behavior (step 303). Statistical bounds may be determined by various statistical methods and/or probability distributions, for instance by a Gaussian distribution. Metrics used to generate the statistical bounds may be weighted differently depending on patient profile. For example, compared with a relatively sedentary patient, a more active patient may more frequently have an elevated heart rate not requiring treatment.

Storage of historical ET sensor data related to arrhythmia or defibrillation events (step 305) may also be desirable for later review. Stored data related to such events may include, for example, date and time of event and/or therapy, signal amplitude, pulse rate, duration of event, number of shocks delivered within a set time period, and efficacy or non-efficacy of therapy. A history of displacement, velocity, acceleration, and/or vector of movement data sensed by an electrode or a set of electrodes may also be stored. Possible instances for information review include by a patient and/or physician on a periodic basis, after an improper shock, or subsequent to removal of the device from the patient.

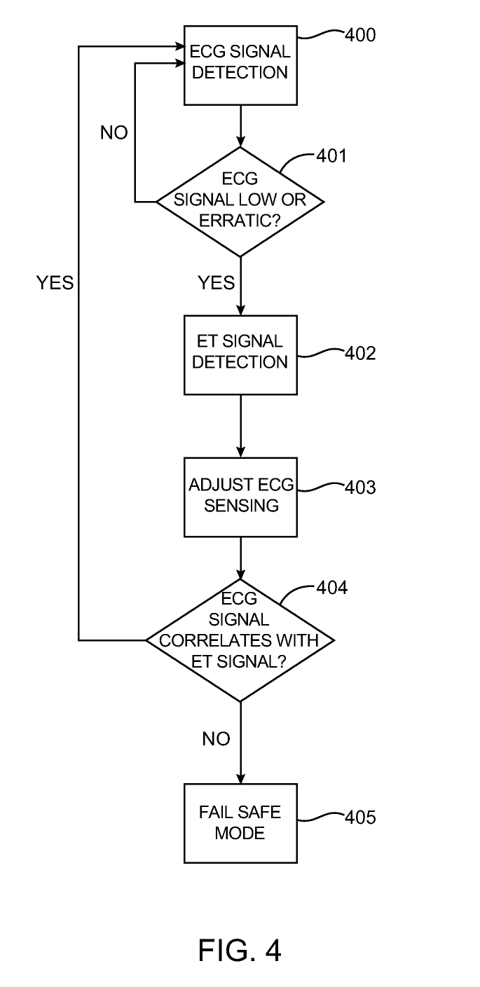

Moreover, ET sensors may contribute functionality to the ET enhanced ICD beyond verification of the ECG signal. For example, in one embodiment of the invention, the ICD control algorithm compares an ET signal and an ECG signal to determine whether the ECG detection system was functioning properly. FIG. 4 is a flow chart illustrating two possible applications of this embodiment of the invention, using ET to adjust ECG signal sensitivity (steps 401-403), and using ET for failsafe operation (step 405). Various embodiments of the invention would feature one or more applications for using an ET signal to verify ECG detection functionality.

In FIG. 4, when an ECG signal exhibits characteristics of faulty detection, e.g., erratic signal, or insufficient sensitivity/oversensitivity, e.g., erratic signal or low signal level, the ICD system adjusts the ECG signal. Adjustment may continue for a set number of cycles until either the ECG signal input correlates with ET signal data or the ECG input is determined to be faulty (steps 401-404). If the ECG detection system is found to have failed, failsafe operation may be initiated (step 405). In one example of failsafe operation, the ET signal could be used in place of the ECG signal to support basic ICD functionality.

Comparison of ET and ECG signals may be used to determine whether a lead has suffered damage, for example, due to lead fracture. For example, if ET data failed to corroborate arrhythmic motion when ECG data indicate the need for a shock, the ICD would not deliver a shock. In this manner, improper shock due to lead damage or failure may be reduced or eliminated.

Continuous Field Tomography in Conjunction with Cardiac Implants

Embodiments of the invention include the use of continuous field tomography in conjunction with cardiac implants, such as pacemakers. The following description provides examples of system configurations that employ continuous field tomography in conjunction with cardiac implants. For ease of description only, the following discussion focuses on electrical tomography embodiments of continuous field tomography. However, it is understood that other embodiments of continuous field tomography, such as magnetic or thermal field tomography, could be similarly used.

During implantation of a pacemaker it is usual for the implanting physician to evaluate the efficacy of a pacing lead using a pacing system analyzer. The pacing system analyzer is a portable electronic device that has a user interface (screen and keyboard, for instance) and a connector for connecting to the pacemaker leads, such as a cable with alligator clips. During the implant procedure, the pacing system analyzer is temporarily attached to the pacemaker lead. The analyzer applies electrical impulses to the lead that are used to evaluate the pacing capture threshold and lead impedance.

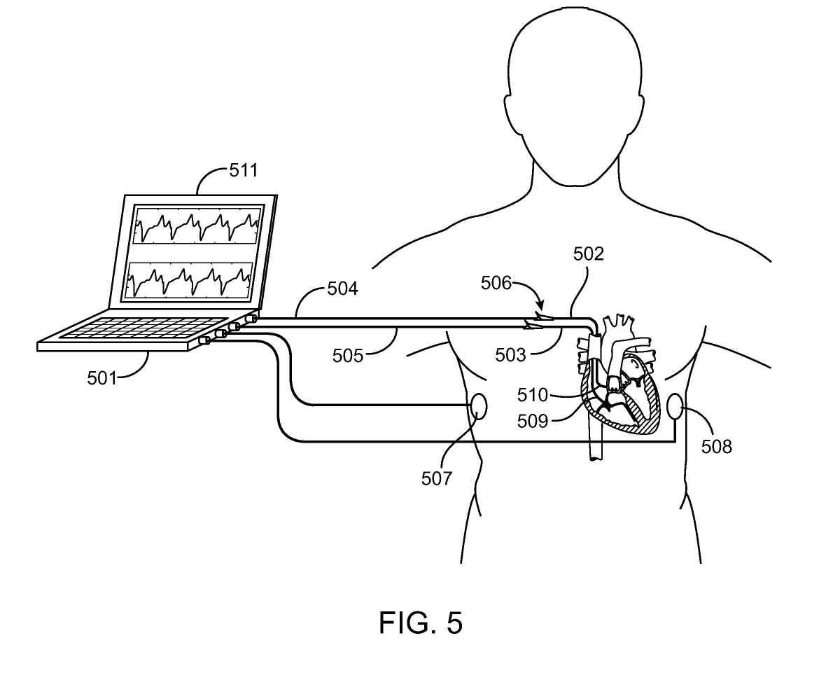

A significant enhancement to a pacing system analyzer is provided by systems of the invention which can simultaneously evaluate pacing performance like a conventional pacing system analyzer and measure cardiac performance using continuous field tomography. Such a device is shown in FIG. 5. Here a console 501 is shown connected to pacing leads 502 and 503 with cables 504 and 505. Alligator clips 506 join the pacing leads and cables temporarily. The console is producing an electrical field across the patient's thorax via skin patches 507 and 508. Only two patches are shown, though in some product configurations there are six patches, such that there are two patches for each of three orthogonal fields. The console 501 provides the functions of generating these electric fields, sensing the resultant electric potentials on the cardiac leads, 509 and 510, and displaying the cardiac motion derived from those potentials on the screen 511. The console provides the function of a pacing system analyzer so that it provides pacing pulses to the leads, it measures the pacing impedance of the leads, and also captures the electrical tomography data.

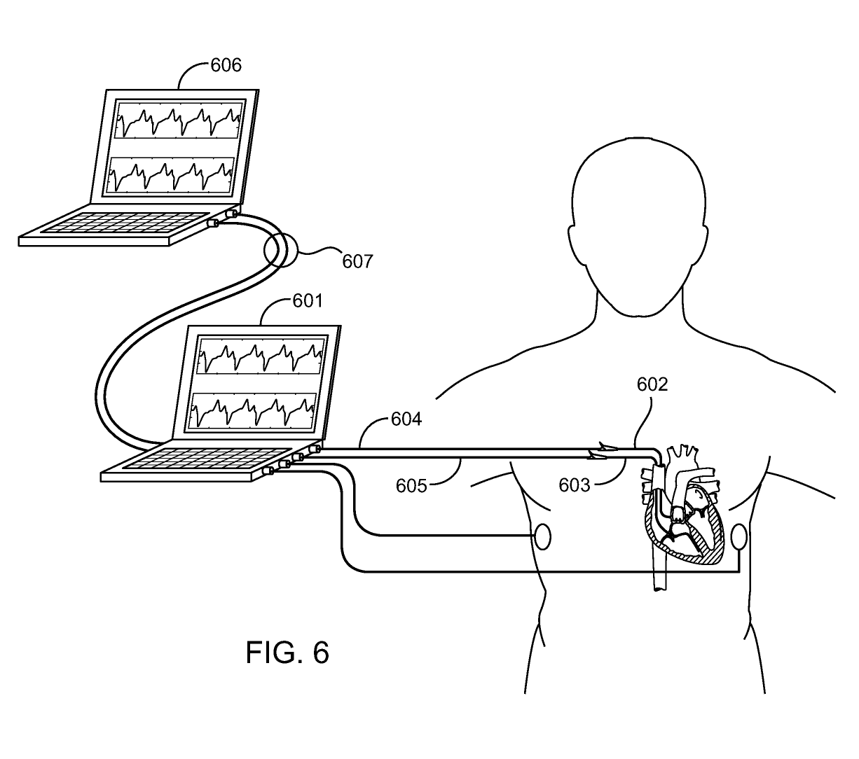

For various commercial, medical or regulatory reasons it may be preferable to separate the pacing system analyzer function from the electrical tomography function. An example of such a system is shown in FIG. 6. Here electrical tomography console 601 is still connected to cardiac leads 602 and 603 via cables 604 and 605. However, in this case the console is a purely diagnostic device, such that it is measuring the electrical tomography signals in order to quantify cardiac performance but is not providing pacing pulses. The pacing pulses are provided by a pace pulse generator 606. This pace pulse generator 606 could be a temporary pacemaker, it could be a pacing system analyzer or it could be a permanent implantable pacemaker. The pace pulse generator 606 is connected to the electrical tomography console via cables 607 in a pass-through mode so that pacing signals from the pacing pulse generator 606 funnel through the electrical tomography console.

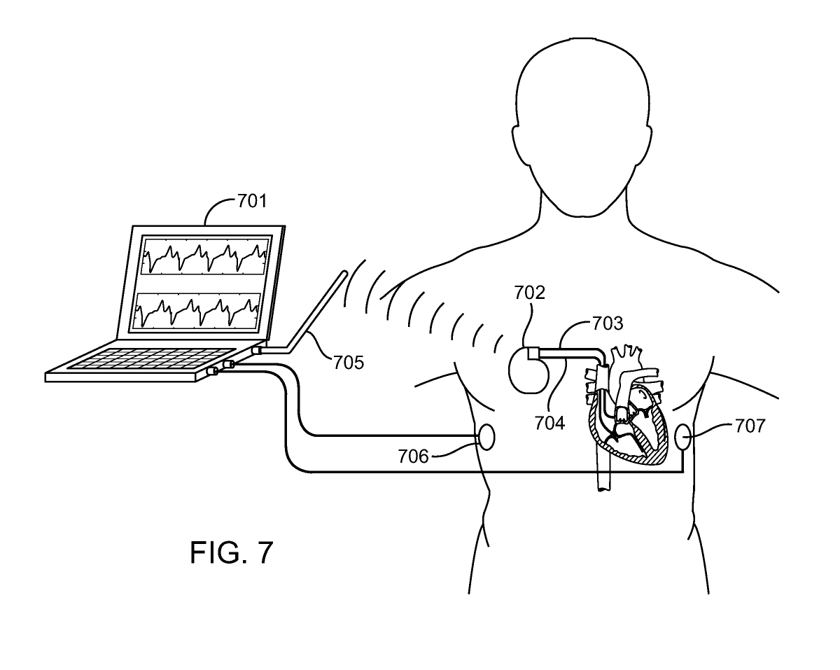

An alternative method for evaluating pacemaker leads at implantation is to connect them to a pacemaker programmer, for example as shown in FIG. 7. Here the console, 701 provides two functions; it again records and displays the electrical tomography signals, but it also is a programmer for an implantable pacemaker. The implantable generator 702 is connected to the pacing leads 703 and 704 and the programmer receives the electrical tomography data via a wireless link, such as antenna 705. The programmer is providing the electric fields for tomography by the skin patches 706 and 707 but is not physically connected to the leads. Because of this, this particular system configuration could be used both at implantation and also during follow up visits.

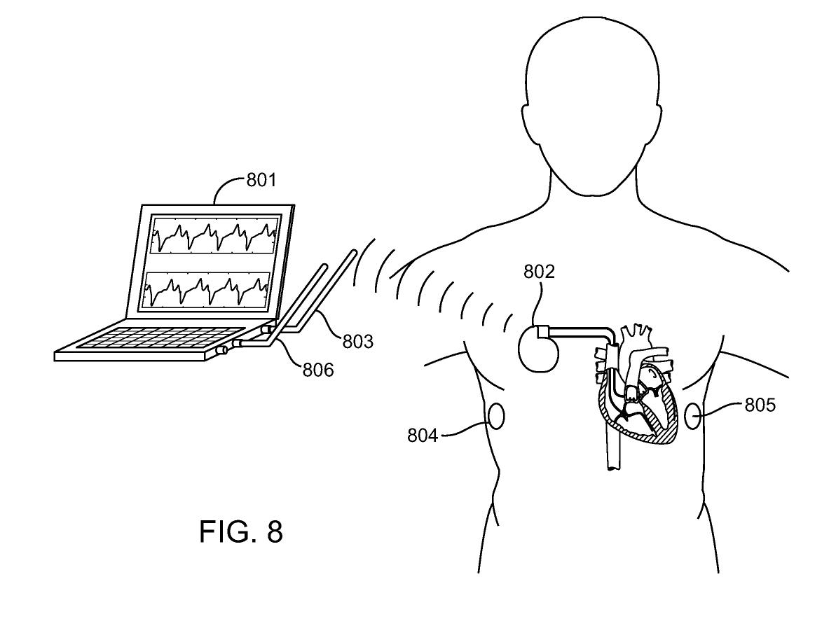

For patient comfort, enhanced electrical safety, and to facilitate measuring cardiac performance during exercise it may be desirable to remove all wired connection between the programmer and the patient. A system according to this embodiment is shown in FIG. 8. In FIG. 8, programmer 801 has both a wireless connection to the implantable generator 802 via antenna 803 and also a wireless connection to the skin patches 804 and 805 via the same or different antenna 806. Skin patches 804 and 805 contain a power source, such as a battery. The skin patches establish an alternating electrical field across the patient's torso. Cardiac leads measure the local electric potential within the heart. Pacemaker 802 processes those electric signals and transmits them to the programmer via a wireless interface. The programmer further processes the signals and displays them in a physiologically meaningful way to the medical professional who may use those results to optimize the pacing parameters using the same console.

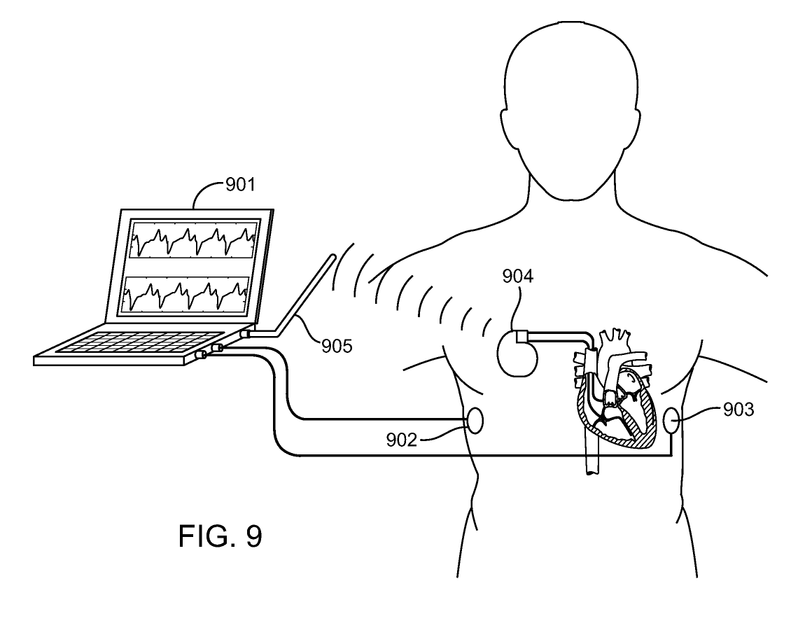

In certain instances it may be desirable to record cardiac performance on a device that does not have the ability to alter the settings of the pacemaker. For instance, a medical professional that does not have pacemaker expertise, such as general practitioner, or the patient themselves, may wish to monitor cardiac performance. Such a device is shown in FIG. 9. Electrical tomography terminal 901 provides the electrical signals via skin patches 902 and 903 and communicates with the implantable pulse generator 904 via a wireless link indicated by antenna 905. Unlike the programmer, this device does not change the pacing settings; it just provides a physician or other healthcare professional a way of monitoring the cardiac performance. This could be used at patient follow-up, it could be used during stress testing (for instance when the patient is on a treadmill) or potentially when the patient is at home.

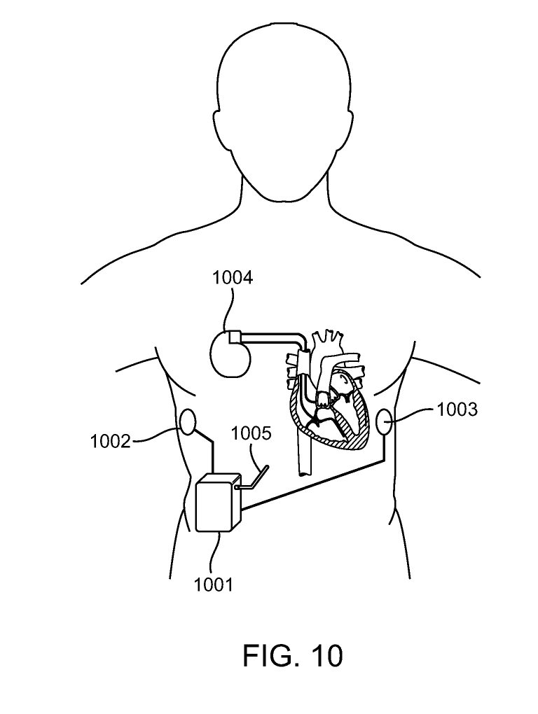

In FIG. 10, the console is reduced to a wearable device with a form factor that is similar to a Holter recorder 1001. The console provides electrical energy to skin patches 1002 and 1003 and communicates wirelessly with the implantable pace generator 1004 via an antenna 1005. In this configuration the patient may wear this device for an extended period in exercise, in their daily routine, and the recorder 1001 would record the cardiac performance over a period of time. This device could be worn for hours, days, weeks or even continuously for an extended period of time.

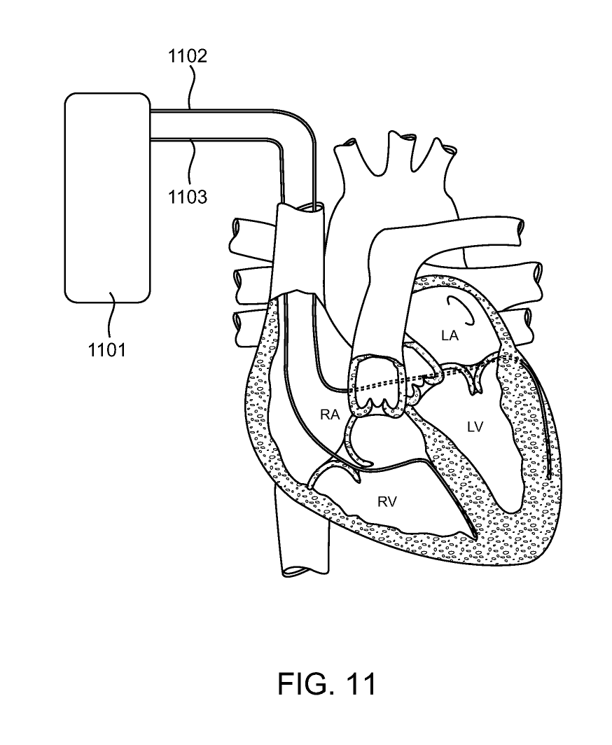

FIGS. 5 to 10 depict the implantable pulse generator as being a cardiac resynchronization therapy (CRT) pacemaker. An alternative configuration is a purely diagnostic implant such as an implantable loop recorder that is shown in item 1101 in FIG. 11. In FIG. 11, the implantable medical device does not generate pacing pulses; it purely records diagnostic information, such as the electrical tomography measure of cardiac performance. The implantable medical device can also potentially measure electrocardiograms. Like a pacemaker, it is connected to an cardiac lead 1102. There could be one or more cardiac leads 1103 to record cardiac motion at additional sites.

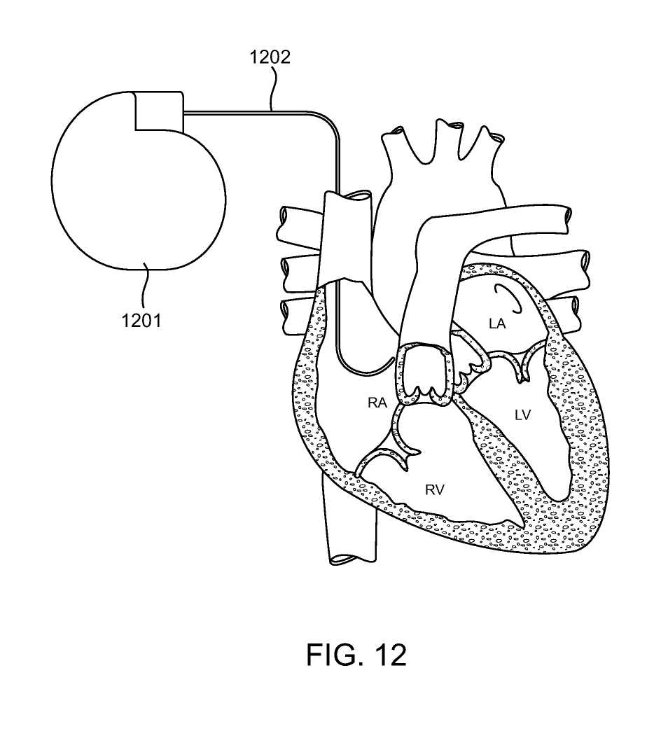

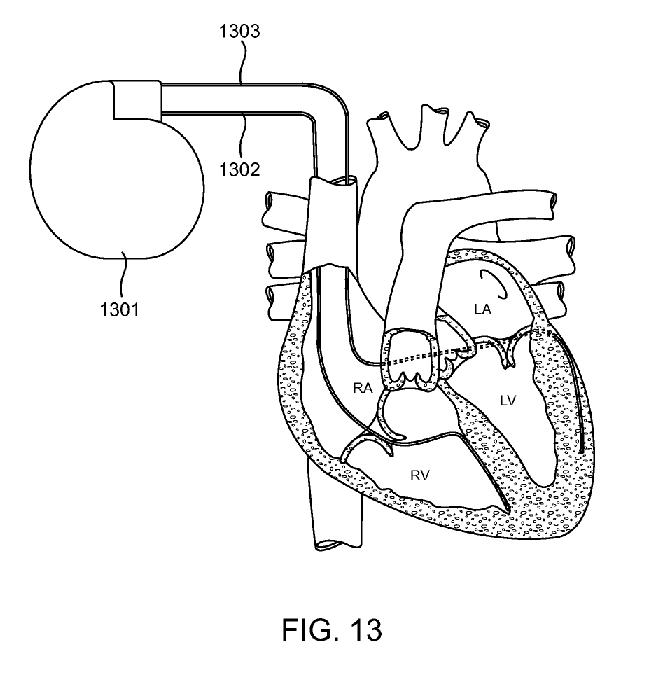

Another system embodiment is shown in FIG. 12. In FIG. 12, the implantable pulse generator is a right atrium only pacemaker. Pace generator 1201 is connected to right atrial pacing lead 1202, and provides electrical stimulation to the right atrium. It also records electrical tomography data from that same lead. Similarly, in FIG. 13, pulse generator 1301 is connected to a right ventricular lead shown here as 1302 to provide right ventricular pacing and also measure cardiac performance derived from the motion of the right ventricular lead.

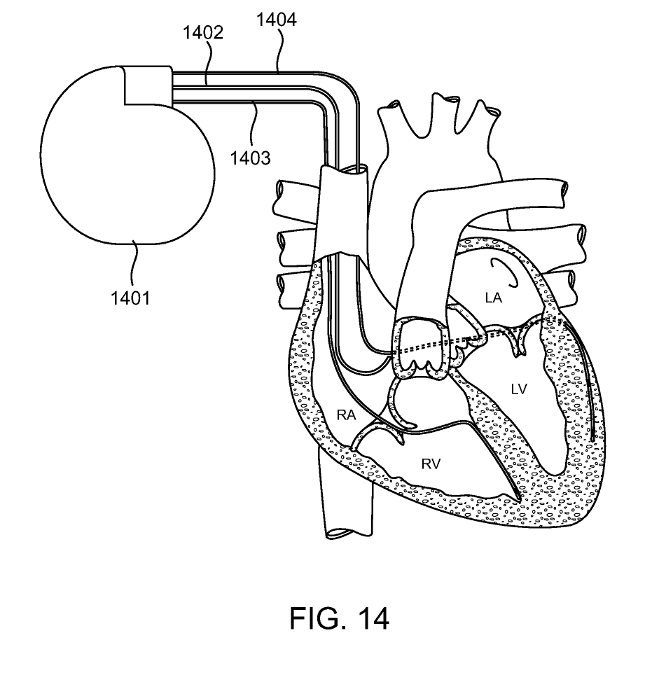

FIG. 14 illustrates a cardiac resynchronization therapy pacemaker that has three leads. The pulse generator 1401 is connected to a right atrial lead 1402, a right ventricular lead 1403 and a left ventricular lead 1404. The depicted leads are conventional unipolar or bipolar pacing leads. The pacemaker provides electrical stimulation to one or more of these leads and measures cardiac motion of the electrodes on the leads.

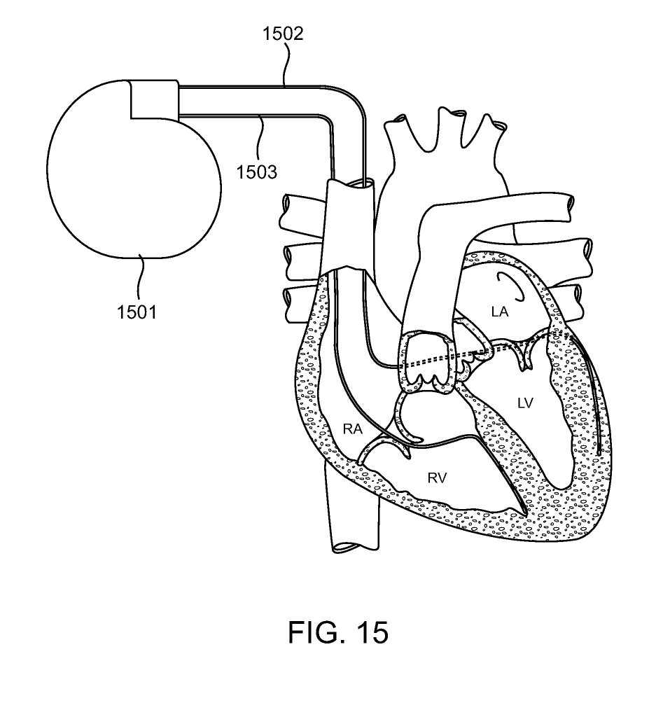

FIG. 15 depicts a device where CRT pace generator 1501 is connected to programmable multi-electrode lead 1502 in the left ventricle and to programmable electrode lead in the right ventricle 1503. The plurality of electrodes on the multi-electrode leads may be used as stimulating electrodes, as electrical tomography signal receiving electrodes, or both. As an example, programmable electrodes on the distal portion of the left ventricular lead may be used for both pacing and measuring the motion of the left ventricle freewall while electrodes more proximal on the same lead may be used solely for measuring the motion of the mitral valve annulus as an indicator of cardiac performance.

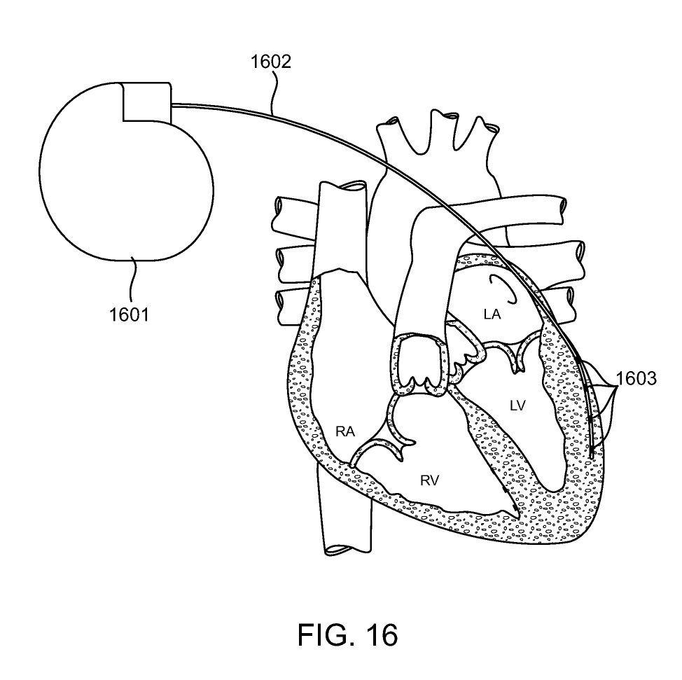

In some cases it is not possible to place a left ventricular lead via a transvenous approach. In this case an epicardial lead is surgically attached to the outer wall of the heart. An electrical tomography cardiac resynchronization pacemaker with an epicardial lead is shown in FIG. 16. Here the cardiac resynchronization pacemaker 1601 is connected to an epicardial left ventricular lead 1602. Epicardial left ventricular lead 1602 a multielectrode lead with a plurality of electrodes on the epicardial surface, shown here as 1603. The electrodes may provide pacing energy that also is being used to measure contractility using electrical tomography and the greater number of electrodes provided by the multielectrode lead provides greater spatial fidelity in the cardiac performance measurement.

### Comprehensive Patient-Related Data Displays

Embodiments of systems and methods of the invention are configured to provide comprehensive patient-related data displays. In various aspects, the comprehensive data are correlated in various manners to provide useful and efficient tools from which accurate diagnoses and inferences may be drawn. The data may be generated by various methods and devices including, for example, continuous field tomography and ingestible event markers. The subject systems and methods find use in a variety of different clinical applications, such as cardiac related applications. The data displays of these embodiments are examples of aggregated data products that may be produced by data aggregating modules of systems of invention.

Cardiac-related applications include, for example, diagnostic and inferential applications predicated on cardiac performance and other metrics. The term “metrics”, as used herein, refers to any measurement, characteristic, property, calculation, or the like, e.g., a measure of tissue location motion, such as of a cardiac tissue location of a heart wall.

In various aspects, data may be generated and aggregated in a variety of ways. For example, multiple data streams from various sources may be combined, various types of data may be combined, etc.

Examples of data generation include continuous field tomography data generation, ingestion information data generation, and patient behavior-related data generation. Continuous filed tomography data may be generated using a variety of protocols, such as described above. Examples of data related to continuous field tomography in general and electrical field tomography specifically (ET data) include stroke volume, ejection fraction, dP/dtₘₐₓ, strain rate, peak systolic mitral annular velocity, end systolic volume end diastolic volume, and QRS length.

Ingestion information, e.g., time of medication ingestion, substance ingested, etc. may be generated via various methods and devices, such as by using the ingestible event markers and systems, as described above. Various IEM data associated with, for example, the ingestible event marker system, include physical data, e.g., data generated by the IEM; derived metrics, e.g., processed physical data to derive various metrics such as time of ingestion data; combined metrics, e.g., derived metrics combined with other derived metric data such as time of ingestion data combined with data identifying the ingested substance; and IEM data, e.g., derived metrics and/or combined metrics aggregated with various physiologic data such as time of ingestion data combined with data identifying the ingested substance and physiologic data such as EKG data, temperature, etc. Ingestion information—related data generation includes generation of data such as medication types, medication dosages, and medication dosage time intervals.

Patient behavior-related data generation includes, for example, electronic and manual recordation of patient-related behavior parameters, e.g., patient decisions regarding ingestion of medication, decision regarding ingestions of various foods, etc. Examples of patient behavior—related metrics include medication ingestion indicators and non-ingestion indicators.

The comprehensive data may be aggregated by a data aggregation module of the invention (such as described above) and then displayed, in whole or in part, via a variety of display devices. Examples include computer devices and non-computer devices. In one physical embodiment a computer device may include a display medium, processor, a memory, a storage medium, and/or various combinations of the same. Examples of computer devices include mobile computers, laptops, desktops, servers, hand-held devices including smart phones and other hand-held computing devices, etc. Examples of non-computer devices include televisions, etc. Various other physical embodiments of computer devices and non-computer devices are possible, as well.

Display modalities include, for example, visual displays, audio displays, etc.

Display media include, for example, visual screen displays, paper printed displays, audio displays via a speaker, etc. In one example, a medical device may display, via a speaker, audio cardiac data as beeps representing a cardiac rhythm and may concurrently display, via a display monitor, visual data such as a cardiac trace.

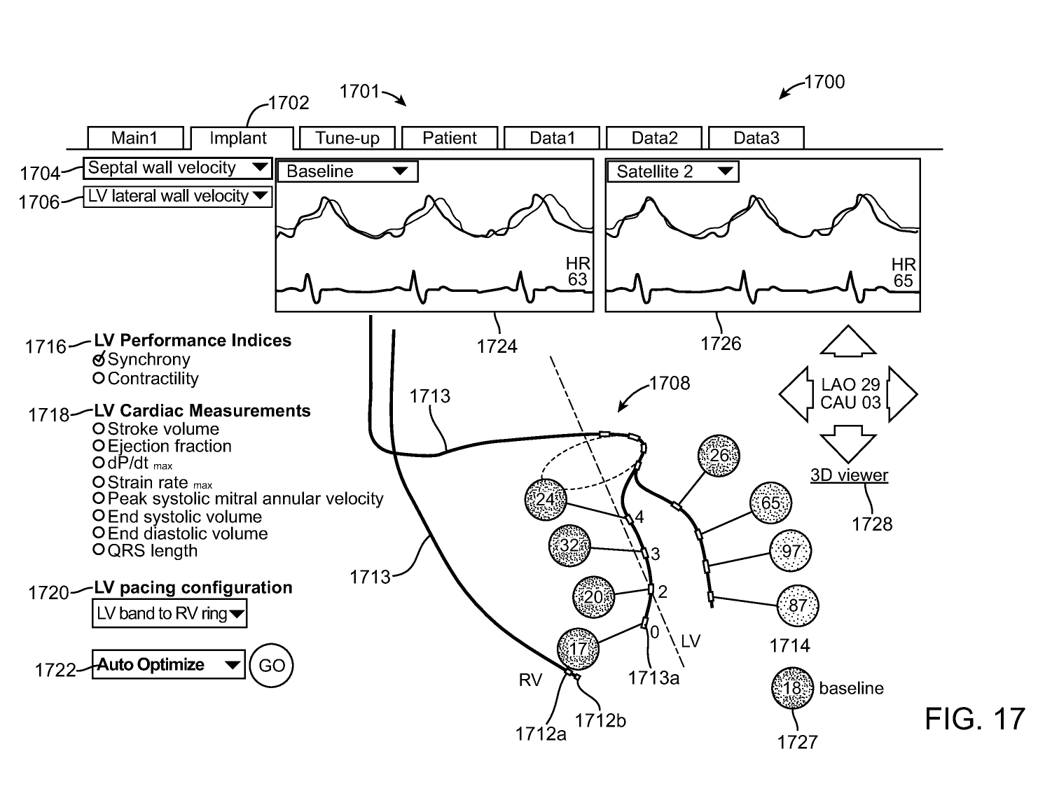

FIG. 17 illustrates a first sample user display 1700, according to an embodiment of the present invention. The first sample user display 1700 may include, for example, tabs 1701 to facilitate selection of various menu choices, e.g., “Main1”, “Implant”, “Tune-up”, “Patient”, “Data1”, “Data2”, and “Data3”. The tab 1702 “Implant”, for example, may be selected to display various data, e.g., septal wall velocity data 1704 and LV lateral wall data 1706.

The various data may be derived, for example, from placement of health devices such as multisensor leads, which may be illustratively displayed in a manner indicative of actual placement. For example, an actual placement GUI 1708 indicates placement of multiple multisensor leads 1712 in the right ventricle (RV) and 1713 left ventricle (LV), respectively.

The actual placement GUI 1708 further indicates the number and relative position of sensors, e.g., RV multisensor lead 1712 having sensor placement identified at locations 1712a, 1712b, respectively, and LV multisensor lead 1713 having sensors 1, 2, 3, and 4 with placements identified at locations 26, 65, 97, and 87, respectively. The LV lead 1713a (shown in phantom) depicts displacement of the LV lead 1713 during, for example, a ventricular contraction. The sensors 1, 2, 3, and 4 are displaced from locations 26, 65, 97, and 87 to placements identified at 24, 32, 20 and 17, respectively.

Various other data may be displayed, e.g., selectively displayed, such as LV performance indices 1716, LV cardiac measurements 1718, LV Pacing Configuration 1720, and an auto optimize option 1722.

The LV performance indices 1716 may provide for selective display of various performance indices such as synchrony, contractibility, etc.

The LV cardiac measurements 1718 may provide for selective display of various parameters, e.g., stroke volume, ejection fraction, dP/dt(max), strain rate(max), peak systolic mitral annular velocity, end systolic volume, end diastolic volume, and QRS length, etc.

The LV pacing configuration 1720 may provide for selective display of various pacing configurations, e.g., LV band to RV ring, etc.

The auto optimize option 1722 may provide for selective automatic optimizing of myocardial stimulation site and device timing parameters.

Still other data may be displayed, e.g., baseline trace 1724 and satellite 2 trace 1726, which may indicate relative utility of one pacing state versus another. The baseline trace 1724 display may be generated, for example, by selecting a baseline option 1727. The satellite trace 1726 may be displayed, for example, by selection of the various data selections.

To illustrate, selection of the LV performance index 1716 “synchrony” may result in display of the relative displacement of the LV multisensor lead sensors 1, 2, 3, and 4, e.g., from an initial placement identified at points 26, 65, 97, and 87 measured at a first point in time to subsequent placements identified at points 24, 32, 20, and 17, respectively, measured at a second point in time. Further, the baseline trace 1724 indicates the degree of dysynchrony in the unpaced state and the satellite trace 1726 indicates the degree of dysynchrony in the paced state, where pacing is occurring via the electrode of sensor 2, as derived from displaced data obtained with sensors 1, 2, 3, and 4. In this manner, for example, the baseline trace 1724 data and the satellite trace 1726 data may be compared. A comparison may indicate, for example, that pacing with satellite 2 results in improved dysynchrony versus the unpaced state.

In addition, the data may be displayed according to various formats, e.g., via selection of a 3D viewer 1728, which displays various data in a three dimensional format.

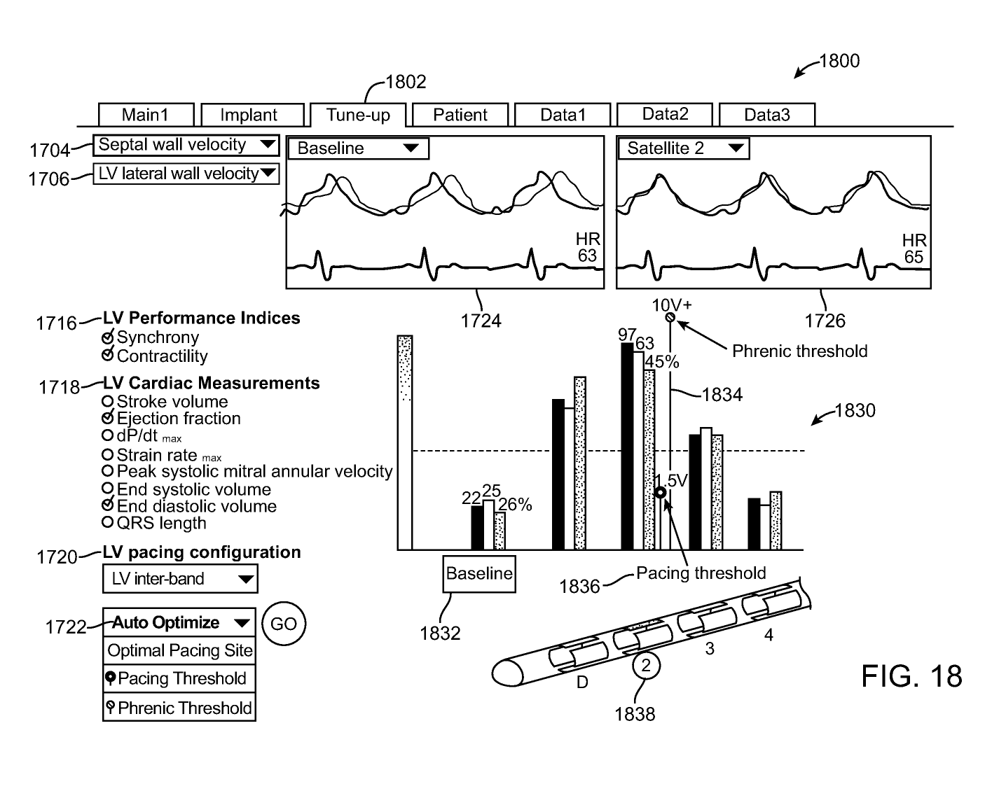

FIG. 18 illustrates a second sample user display 1800, according to an embodiment of the present invention. The second sample user display 1800 includes, for example, the tab 1802 “tune-up” selected to display various data, e.g., the septal wall velocity data 1704 and the LV lateral wall data 1706 in relation to both synchrony and contractility performance indices.

Lead sensor indicator 1838 may indicate with which sensor, e.g., sensor 2, the data is associated.

Graph 1830 may provide for data associated with various indices and parameters.

To illustrate, selection of the LV performance indices 1716 “synchrony” and “contractility” as well as the LV cardiac measurement 1718 “ejection fraction” and LV pacing configuration 1720 “LV Inter-band” and auto optimize option 1722 set for “phrenic threshold” may result in display of the graph 1830 having baseline indicators 1832, phrenic threshold indicator 1834, pacing threshold 1836, etc. In this manner, a viewer may quickly and accurately receive and assess various patient-related data.

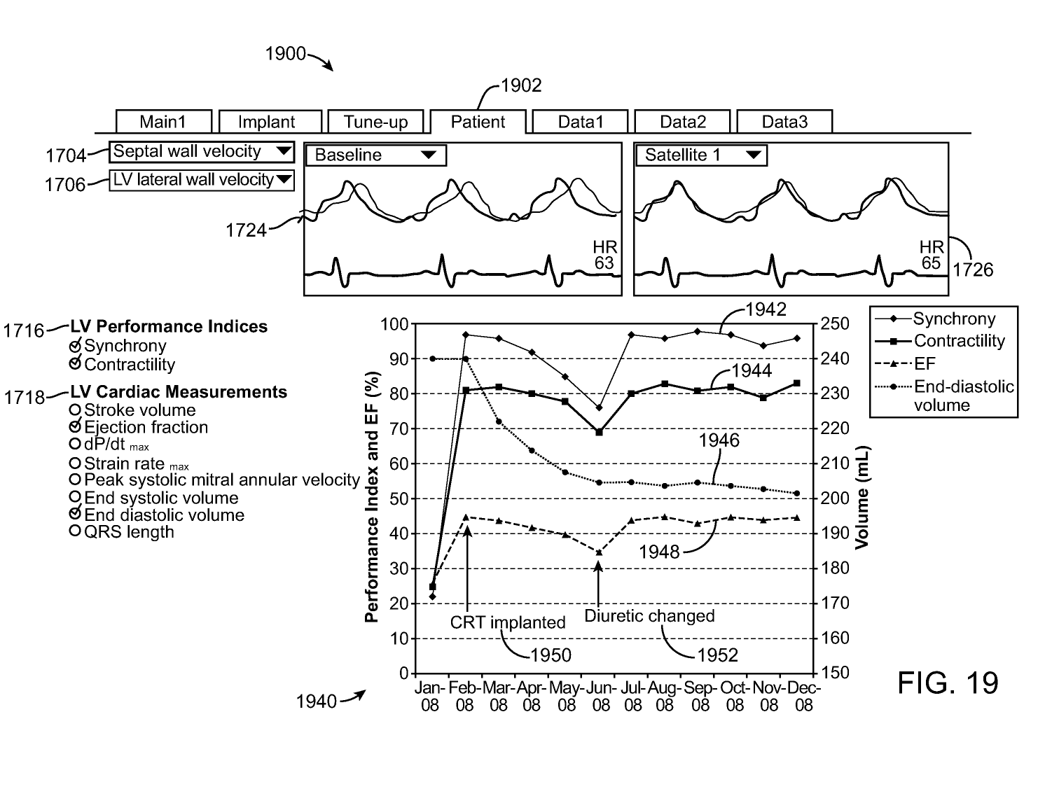

FIG. 19 illustrates a third sample user display 1900, according to an embodiment of the present invention. The sample user display 1900 includes, for example, the tab 1902 “Patient” selected to display various data, e.g., the septal wall velocity data 1704 and the LV lateral wall data 1706 in relation to synchrony and contractility performance indices over time.

Graph 1940 may provide data associated with various indices and parameters.

To illustrate, selection of the LV performance indices 1716 “synchrony” and “contractility” as well as the LV cardiac measurements 1718 “ejection fraction” and “end diastolic volume” may result in synchrony trace 1942, contractility trace 1944, end-diastolic volume trace 1946, and ejection fraction (EF) trace 1948, respectively, according to performance index and EF percentage (y-axis) over time (x-axis).

The synchrony trace 1942 may indicate, for example, an initial improvement in dysynchrony immediately after CRT implant, then a transient worsening of dysynchrony in May-June, followed by stabilization of the degree of dysynchrony.

The contractility trace 1944 may indicate, for example, an initial improvement in contractility immediately after CRT implant, then a transient worsening of contractility in May-June, followed by stabilization of contractility.

The ejection fraction (EF) trace 1948 may indicate, for example, an initial improvement in EF immediately after CRT implant, then a transient worsening of EF in May-June, followed by stabilization of the EF.

The end-diastolic volume trace 1946 may indicate, for example, steady improvement toward eventual stabilization over time.

Time correlation with specific events may provide further diagnostic/inferential data. For example, CRT implanted event 1950 in February 2008 correlates to spikes, i.e., increase in performance indices and EF percentages, in each of the traces synchrony trace 1942, contractility trace 1944, and EF trace 1948. The CRT implanted event 1950 further correlates to a decrease in the end-diastolic trace 1946.

From these, it may be inferred, for example, that the patient's overall cardiac performance improved following CRT implantation, aside from a transient worsening of dysynchrony, contractility, and EF in May-June. Similarly, data associated with a medication change event, e.g., diuretic changed event 1952 in June 2008, may be received from various sources and patient behavior-related data sources.

The diuretic changed event 1952 in June 2008 may temporally correspond to a reverse in the trend of decrease over time to an immediate increase, i.e., in the interval between June 2008 and July 2008, in the synchrony trace 1942, the contractility trace 1944, and the EF trace 1948. The end-diastolic volume trace 1946 continued its decrease over time, although at decreasing rate, as compared with the entire period preceding the diuretic changed event 1952.

From these, it may be inferred, for example, that the patient was on a suboptimal medical regimen that contributed to the transient worsening in dysynchrony, contractility, and EF, in May-June and that the worsening trend was reversed after a change in the diuretic component of the medical regimen.

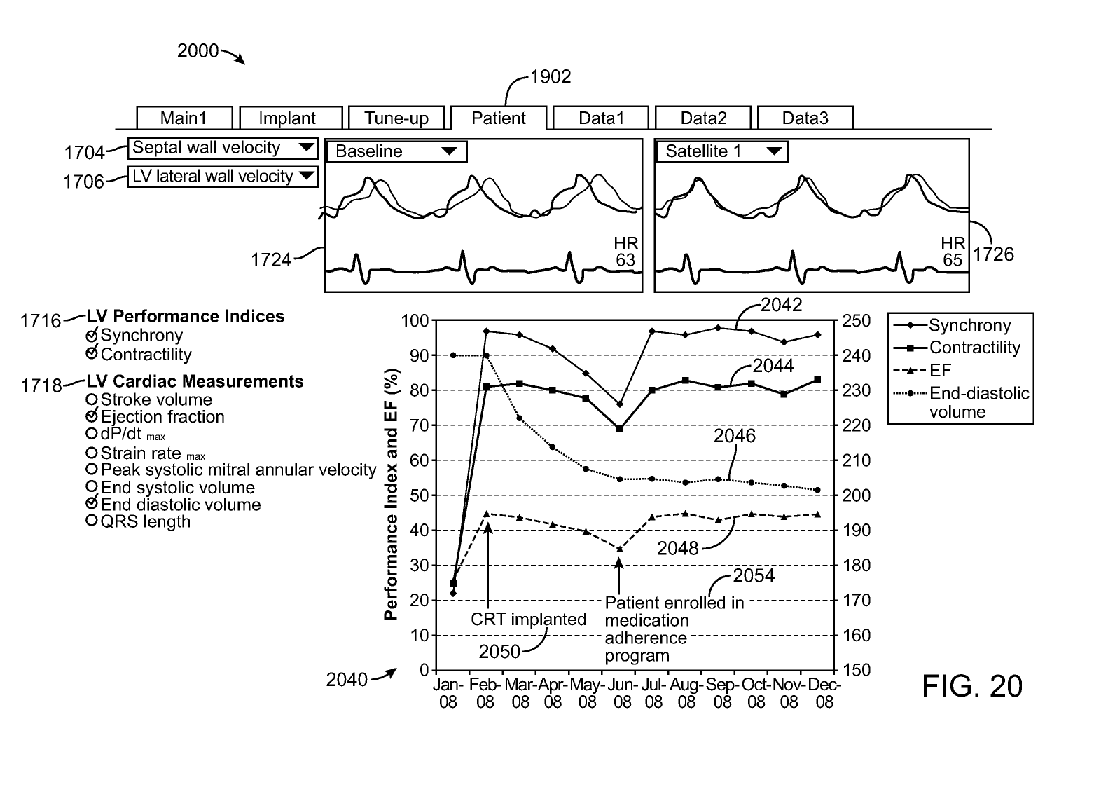

FIG. 20 illustrates a fourth sample user display 2000, according to an embodiment of the present invention. The fourth sample user display 2000 may include, for example, the CRT implanted event 2050 in February 2008 and a patient enrolled in medication adherence program event 2054. The enrollment of the patient in the medication adherence program may include, for example, interaction with patient behavior-related data generation methods and devices.

To illustrate, the patient has the CRT implanted in February 2008 and is started on a corresponding treatment regimen including medication and dietary therapies. Tracking of patient cardiac parameters synchrony, contractility, ejection fraction, and end-diastolic volume, via, for example, ET methods generating related data and displaying via synchrony trace 2042, contractility trace 2044, end-diastolic volume trace 2046, and ejection fraction (EF) trace 2048, respectively, indicate an overall degradation in cardiac performance during the interval February 2008 to June 2008. The patient parameters may be routinely provided to the patient, the patient's physician, and the family caregiver via various means.

Upon analysis of the patient parameters, the physician enrolls the patient in the medication adherence program, which tracks sentinels for wellness including medicine therapy, e.g., medication ingestion. In addition, sentinels for wellness including cardiac parameters, blood pressure, and weight are also tracked.

Prior to enrollment, the patient behavior-related data indicates that the patient has neglected to take the medication at the appropriate times. Subsequent to enrollment, various behavior modifications on the part of the patient result in timely medication ingestion which, in turn, result in changes in the sentinels for wellness. The changes may be reported to the parties via the synchrony trace 2042, contractility trace 2044, and EF trace 2048, during the interval June 2008 to December 2008. From the traces, the patient, physician, and family caregivers may be able to visually note an improvement over time and are able to continue to monitor progress. In this manner, display of aggregated data from various sources provide the tools necessary to quickly and accurately assess, diagnose, and infer various clinical data related to a patient.

Continuous Field Tomography in Conjunction with Additional Diagnostic Modalities

Aspects of the invention include using continuous field tomography obtained data, e.g., as described above, with one or more additional diagnostic modalities.

Electric Tomography can be combined with numerous imaging and other diagnostic systems to provide a pooled, complementary data set to enhance clinical decision making. In this circumstance, ET data can be gathered either simultaneously or sequentially with data collection by the other system. Furthermore, ET functionality can be physically integrated into or physically separate from the other system. Finally, the data acquired by both systems can be presented to the user side-by-side, sequentially, or in a visually super-imposed manner.

In some embodiments, ET data is aggregated by the data aggregator module with cardiac scintigraphy data. Cardiac scintigraphy evaluates myocardial perfusion and/or function to detect physiologic and anatomic abnormalities of the heart. There are five major classes of cardiac scintigraphy: myocardial perfusion imaging, gated cardiac blood-pool imaging, first-pass cardiac imaging, myocardial infarction imaging, and right-to-left shunt evaluation (American College of Radiology Standard for the Performance of Cardiac Scintigraphy). In cardiac scintigraphy, a subject is administered an radioisotopic label and the heart is imaged to obtain the cardiac scintigraphic data. Scintigraphy visually indicates myocardial regions that are ischemic or infracted. ET, via differential motion signals generated from electrodes along the myocardium, indicates areas of akinesis/hypokinesis. If concordant with scintigraphy results, ET increases the specificity of the combined diagnostic test.

In some instances, ET data is aggregated with electrocardiography data to produce an aggregated data product. An echocardiogram is a test in which ultrasound is used to examine the heart. In addition to providing single-dimension images, known as M-mode echo that allows accurate measurement of the heart chambers, the echocardiogram also offers two-dimensional (2-D) Echo and is capable of displaying a cross-sectional “slice” of the beating heart, including the chambers, valves and the major blood vessels that exit from the left and right ventricle. Doppler is a special part of the ultrasound examination that assesses blood flow (direction and velocity). In contrast, the M-mode and 2-D Echo evaluates the size, thickness and movement of heart structures (chambers, valves, etc.). During the Doppler examination, the ultrasound beams will evaluate the flow of blood as it makes it way though and out of the heart. This information is presented visually on the monitor (as color images or grayscale tracings and also as a series of audible signals with a swishing or pulsating sound).

Echocardiography provides important information about, among other structures and functions, the size of the chambers of the heart, including the dimension or volume of the cavity and the thickness of the walls. The appearance of the walls may also help identify certain types of heart disease that predominantly involve the heart muscle. Pumping function of the heart can also be assessed by echocardiography. One can tell if the pumping power of the heart is normal or reduced to a mild or severe degree. This measure is known as an ejection fraction or EF. A normal EF is around 55 to 65%. Numbers below 45% usually represent some decrease in the pumping strength of the heart, while numbers below 30 to 35% are representative of an important decrease. Echocardiography can also identify if the heart is pumping poorly due to a condition known as cardiomyopathy, or if one or more isolated areas have depressed movement due to prior heart attacks. Thus, echocardiography can assess the pumping ability of each chamber of the heart and also the movement of each visualized wall. The decreased movement, in turn, can be graded from mild to severe. In extreme cases, an area affected by a heart attack may have no movement (akinesia), or may even bulge in the opposite direction (dyskinesia). The latter is seen in patients with aneurysm of the left ventricle or LV.

Echocardiography identifies the structure, thickness and movement of each heart valve. It can help determine if the valve is normal, scarred from an infection or rheumatic fever, thickened, calcified, torn, etc. It can also assess the function of prosthetic or artificial heart valves. The additional use of Doppler helps to identify abnormal leakage across heart valves and determine their severity. Doppler is also very useful in diagnosing the presence and severity of valve stenosis or narrowing. Unlike echocardiography, Doppler follows the direction and velocity of blood flow rather than the movement of the valve leaflets or components. Thus, reversed blood direction is seen with leakages while increased forward velocity of flow with a characteristic pattern is noted with valve stenosis.

Echocardiography is used to diagnose mitral valve prolapse (MVP), while Doppler identifies whether it is associated with leakage or regurgitation of the mitral valve (MR). The volume status of blood vessels can also be monitored by echocardiography. Low blood pressure can occur in the setting of poor heart function but may also be seen when patients have a reduced volume of circulating blood (as seen with dehydration, blood loss, use of diuretics or “water pill”, etc.). In many cases, the diagnosis can be made on the basis of history, physical examination and blood tests. However, confusion may be caused when patients have a combination of problems. Echocardiography may help clarify the confusion. The inferior vena cava (the major vein that returns blood from the lower half of the body to the right atrium) is distended or increased in size in patients with heart failure and reduced in caliber when the blood volume is reduced. Echocardiography is useful in the diagnosis of fluid in the pericardium. It also determines when the problem is severe and potentially life threatening. Other diagnoses made by Doppler or echocardiography include congenital heart diseases, blood clots or tumors within the heart, active infection of the heart valves, abnormal elevation of pressure within the lungs, among others.

The aggregated data product of echocardiography and ET data may be employed in a number of different ways. For example, motion data from ET may be used to inform the 3D translation and rotation of images generated by echochardiography. Flow data from echochardiography may be combined with ET motion data to increase the specificity of the diagnosis of diastolic dysfunction. In yet other instances, ET data may be calibrated with stress echocardiography data by matching areas of hypokinesis, so that an implantable ET system could be used for longitudinal monitoring of the identified areas of ischemia. For example, ET data may be aggregated with data obtained from an exercise treadmill test. A treadmill test, using ECG signals, provides a non-localizable indication of cardiac ischemia. The test has relatively low specificity. ET, via differential motion signals generated from electrodes along the myocardium, indicates areas of akinesis/hypokinesis. If ET data is employed concordantly with treadmill results, the aggregated data product increases the specificity of the combined diagnostic test. In yet other embodiments, ET data is aggregated with data obtained from ECG/external rhythm monitors (bedside and ambulatory). ECG/external rhythm monitors are the gold standard for diagnosis of dysrhythmias. However, there is sometimes ambiguity to the electrocardiographic diagnosis. For instance, ventricular tachycardia can easily be confused with supraventricular tachycardia (originating above the ventricle) with aberrant conduction, and the latter has far less grave clinical implications. ET motion data can differentiate between atrial and ventricular dysrhythmias, so these mechanical measures would increase the specificity of the diagnostic test.

ET data may also be aggregated with fluoroscopic data to provide an aggregated data product. Fluoroscopy is a process for obtaining continuous, real-time images of an interior area of a patient via the application and detection of penetrating X-rays. Put simply, X-rays are transmitted through the patient and converted into visible spectrum light by some sort of conversion mechanism (e.g., X-ray-to-light conversion screen and/or X-ray image intensifier). Subsequently, the visible light is captured by a video camera system (or similar device) and displayed on a monitor for use by a medical professional. More recently, a solid-state pixelized flat panel is used for this purpose. Typically, this is done to examine some sort of ongoing biological process in the human body, e.g., the functioning of the lower digestive tract or heart. Fluoroscopy is used during cardiac catheterization to provide a semi-quantitative assessment of cardiac contractility (an “LV gram”, requiring a large bolus of intravenous contrast material). ET provides data about global and local myocardial motion data that, when aggregated with fluoroscopic data, corroborates or obviates the LV gram.

ET data may also be aggregated with computed tomography data to provide an aggregated data product. In computed tomography (CT) imaging systems, an x-ray source emits a fan-shaped x-ray beam toward a subject or object, such as a patient, positioned on a support. The beam, after being attenuated by the subject, impinges upon a detector assembly. The intensity of the attenuated x-ray beam received at the detector assembly is typically dependent upon the attenuation of the x-ray beam by the subject. Each detector element of the detector assembly produces a separate electrical signal indicative of the attenuated x-ray beam received. In known third generation CT systems, the x-ray source and the detector assembly are rotated on a rotatable gantry portion around the object to be imaged so that a gantry angle at which the fan-shaped x-ray beam intersects the object constantly changes. Data representing the strength of the received x-ray beam at each of the detector elements is collected across a range of gantry angles. The data are ultimately processed to form an image of the object. In some instances, collection of CT data includes collection of positron emission tomography (PET) data, such that the CT data may be viewed as CT/PET data. CT is used to evaluate calcification in coronary arteries, which is relatively non-specific for clinically significant coronary artery disease. ET, via differential motion signals generated from electrodes along the myocardium, indicates areas of akinesis/hypokinesis. If ET data is concordant with CT results, ET increases the specificity of the combined diagnostic test.

ET data may also be aggregated with magnetic resonance imaging data to provide an aggregated data product. In magnetic resonance imaging (MRI), pulse sequences consisting of RF and magnetic field gradient pulses are applied to an object (a patient) to generate magnetic resonance signals which are scanned in order to obtain information therefrom and to reconstruct images of the object. The pulse sequence which is applied during a MRI scan determines the characteristics the reconstructed images, such as location and orientation in the object, dimensions, resolution, signal-to-noise ratio, contrast, sensitivity for movements, etc. MRI is being used increasingly to provide functional cardiac data, including ejection fraction, regional strain, the degree of valvular regurgitation, and the area of ischemia or infarct. When ET data is aggregated with MRI data in accordance with the invention, ET data can provide proxy of these measures, and therefore may corroborate and increase the specificity of MR measures.

ET data may also be aggregated with pulmonary artery catheter (PAC) data to provide an aggregated data product. Pulmonary artery catheters (“PACs”) are widely used for patient diagnosis and for hemodynamic and therapeutic monitoring. One of the most widely used PACs is the Swan-Ganz catheter. The Swan-Ganz catheter includes a flexible tube (enclosing multiple lumina) that is designed to be flow-directed through a patient's heart by a distal balloon. The catheter is adapted to be delivered through the right atrium and right ventricle with the distal end positioned within the pulmonary artery. The Swan-Ganz catheter includes first and second lumina for use in measuring blood pressures in the pulmonary artery and right atrium respectively. A third lumen is used for inflating the balloon at the distal end of the catheter. fourth lumen is included for housing a thermistor that is used in monitoring blood temperature and in determining cardiac output. A fourth lumen also houses the wires associated with electrodes that are included for monitoring intraatrial and intraventricular electrograms. The Swan-Ganz catheter has been a useful tool in diagnosing complex cardiac arrhythmias. A PAC can provide numerous indices of cardiac performance (cardiac output, right-sided heart pressures, mixed venous O2 saturation). With PACs, Indwelling time is limited by the risk of infection. Simultaneous ET and PAC data may be used in some instances to calibrate ET's proxy measures of these indices. Following this aggregation step, the PAC may be removed and ongoing monitoring of the patient may occur using the resultant PAC calibrated ET system alone, without the added risk of infection.

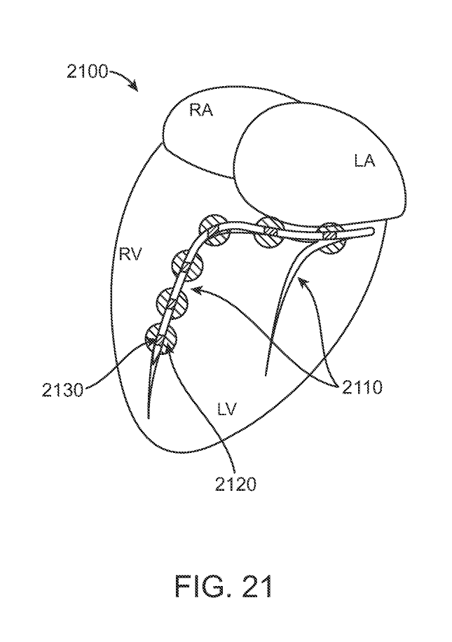

FIGS. 21 to 23 provide three different views of ET data and data obtained from an additional diagnostic modality may be aggregated into an aggregated data product and then presented to a user, for example via a GUI, according to embodiments of the invention.

In FIG. 21, an image 2100 of a heart as determined via computed tomography is shown. The image 2100 may be a live image (such as viewed in a catheter lab) or an image obtained at a prior time. The image shows the right atrium (RA), left atrium (LA), right ventricle (RV) and left ventricle (LV). Also shown are coronary veins 2110. Also shown in the image are ET generated electrode map locations 2120 which are obtained from an ET multiplexed multi-electrode lead 2130, for example as described above. ET generated electrode map locations 2120 may be color or size coded to indicated a number of parameters of interest, such as velocity, timing or direction. For example, a red map location may be employed to indicate movement into the image while a blue map location may be employed to indicate movement out of the image.

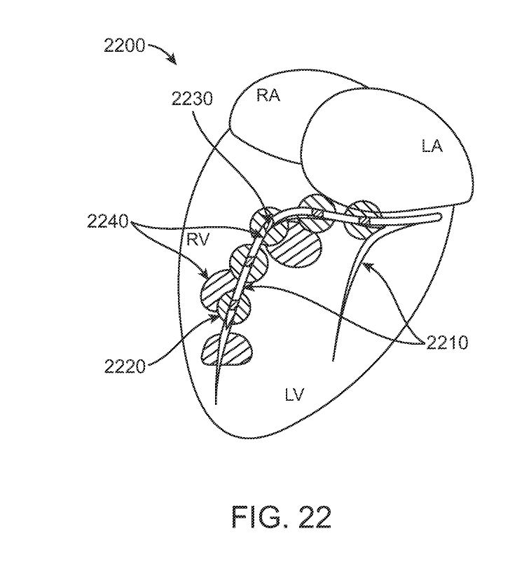

In FIG. 22, an image 2200 of a heart as produced from CT/PET data is provided. The image shows the right atrium (RA), left atrium (LA), right ventricle (RV) and left ventricle (LV). Also shown are coronary veins 2210. Also shown in the image are ET generated electrode map locations 2220 which are obtained from an ET multiplexed multi-electrode lead 2230, for example as described above. ET generated electrode map locations 2220 may be color coded to indicated a number of parameters of interest, such as velocity, timing or direction. Also shown are infarct regions 2240 which are determined from the CT/PET data. From the aggregated data product which is image 2200, a user may identify ET regions of lower velocity and late timing and employ these regions for pacing. A user may also identify overlapping regions of ET map locations and infarct regions as regions to avoid for pacing.

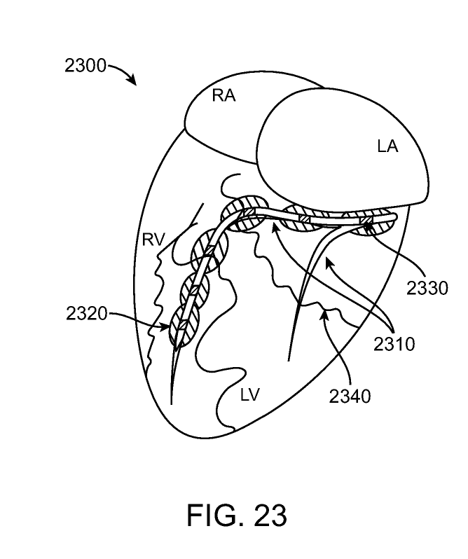

In FIG. 23, an image 2300 of a heart as produced from CT data is provided. The image shows the right atrium (RA), left atrium (LA), right ventricle (RV) and left ventricle (LV). Also shown are coronary veins 2310. Also shown in the image are ET generated electrode map locations 2320 which are obtained from an ET multiplexed multi-electrode lead 2330, for example as described above. ET generated electrode map locations 2320 may be color coded to indicate a number of parameters of interest, such as velocity, timing or direction. Also shown ECG identified heart image elements 2340.

### Electrical Tomography for Use in Implanted Medical Device Location Assessment

In some instances, ET data is employed with implanted medical devices to confirm proper location of the implanted medical device. As such, ET data is employed to detect dislodgement of an implanted medical device. Examples of medical devices for which ET data may be employed to confirm proper positioning or dislodgement include, but are not limited to: cochlear implants; orthopedic implants, such as spinal fixation devices, inter-vertebral disc implants, hip implants, and knee implants; ocular retinal implants; and ear/nose/throat implants.

Implantable devices may or may not include one or more electrodes which are employed in obtaining ET data about the devices. Some implant devices, such as cochlear implants, already have electrodes which can be used with ET to track position of the electrodes. Other implant devices, such as spine fixation implants, may be readily modified to include any electrodes and electronics so that their location can be determined via ET.

As reviewed above, ET methods may employ external or internal electrical fields. Skin electrode patches placed on the skin around the implant may be used to generate the external electrical fields. The electrodes on the implant and other implanted devices may be used to generate internal electrical fields.

A dislodgement of the implant can be detected by measuring the baseline position of the implant at the time of implantation using ET and then comparing it with follow-up ET measurements. If the implant device becomes detached from its original implant location, ET measurements will show a change in position indicative of dislodgement. This change will be useful information in notifying physician for the need for re-implantation and will be useful in diagnosing the root cause of potential non-responders or complications in the case of a dislodgement of the implanted device.

In some instances, this ET data may be aggregated with additional data obtained from the implant, such as how the implant is performing, etc. In these instances, the ET data is aggregated with non-ET implant derived data to produce an aggregated data product.

In some instances, no aggregation occurs, such that ET is employed alone with an implant configured to be monitored via ET in order to simply determine proper implant positioning or dislodgement.

### Methods

Aspects of various method embodiments of the invention have been described above. In some instances, methods of the invention include receiving continuous field tomography data and non-continuous field physiological data at a system that includes a data aggregating module, such as described above. Such methods may include obtaining the continuous field tomography data and/or the non-continuous field physiological data. In some instances, the methods will also include outputting the aggregated data product to another device, such as an implantable medical device, or to a user, for example by displaying the aggregated data product a user.

Also provided are methods that include forwarding continuous field tomography data and non-continuous field physiological data to a system that includes a data aggregating module, such as described above. Such methods may include obtaining the continuous field tomography data and/or the non-continuous field physiological data that is forwarded to the system. In certain of these embodiments, the methods include receiving an aggregate data product from the system, e.g., where the aggregated data product is displayed on a display unit and the method comprises viewing the aggregate data product. In some instances, the methods include modifying an operational parameter of a medical device in response to receiving the aggregate data product, for example where the medical device is an implantable medical device, such as a cardiac device.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

### Computer Readable Medium

One or more aspects of the subject invention may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer readable medium may include stored programming embodying an algorithm for carrying out the steps performed by the data aggregating module. Accordingly, such a stored algorithm includes instructions that, when executed by a computing platform, result in execution of a method of: receiving continuous field tomography data; receiving non-continuous field physiological data; and producing an aggregated data product from the received continuous field tomography data and non-continuous field physiological data.

Of particular interest in certain embodiments are systems loaded with such computer readable mediums such that the systems include a data aggregating module of the invention.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A system comprising:
a data aggregating module configured to:
receive continuous field tomography data;
receive non-continuous field physiological data; and
produce an aggregated data product from the received continuous field tomography data and non-continuous field physiological data.

2. The system according to claim 1, wherein the continuous field tomography data is electric tomography data.

3. The system according to claim 1, wherein the aggregated data product comprises information that is configured to be employed by a user.

4. The system according to claim 3, wherein the aggregated data product is configured to be displayed to a user.

5. The system according to claim 4, wherein the aggregated data product is configured to be displayed to a user on an image display device.

6. The system according to claim 1, wherein the aggregated data product comprises information that is to be employed by a device.

7. The system according to claim 1, wherein the device is configured to modify an operating parameter in response to receiving the information.

8. The system according to claim 1, wherein the system further comprises a continuous field tomography data source

9. The system according to claim 1, wherein the system further comprises a non-continuous field physiological data source.

10. The system according to claim 9, wherein the non-continuous field physiological data source comprises an implantable device.

11. The system according to claim 10, wherein the implantable device is a cardiac device.

12. The system according to claim 9, wherein the non-continuous field physiological data source comprises an extra-corporeal body-associated device.

13. The system according to claim 12, wherein the extra-corporeal body-associated device comprises a conductively transmitted signal receiver.

14. The system according to claim 9, wherein the non-continuous field physiological data source comprises an ingestible event marker.

15. The system according to claim 9, wherein the non-continuous field physiological data source comprises a patient-related behavior parameter recordation device.

16. A method comprising:
receiving continuous field tomography data and non-continuous field physiological data at a system comprising a data aggregating module configured to:
receive continuous field tomography data;
receive non-continuous field physiological data; and
produce an aggregated data product from the received continuous field tomography data and non-continuous field physiological data.

17. The method according to claim 16, wherein the method further comprises obtaining the continuous field tomography data.

18. The method according to claim 16, wherein the method further comprises obtaining the non-continuous field physiological data.

19. The method according to claim 16, wherein the method further comprises outputting the aggregated data product.

20. The method according to claim 19, wherein the aggregated data product is output to an implantable medical device.

21. The method according to claim 19, wherein the aggregated data product is displayed to a user.

22. A method comprising:
forwarding continuous field tomography data and non-continuous field physiological data to a system comprising a data aggregating module configured to:
receive continuous field tomography data;
receive non-continuous field physiological data; and
produce an aggregated data product from the received continuous field tomography data and non-continuous field physiological data.

23. The method according to claim 22, wherein the method further comprises obtaining the continuous field tomography data.

24. The method according to claim 22, wherein the method further comprises obtaining the non-continuous field physiological data.

25. The method according to claim 22, wherein the method further comprises receiving the aggregated data product.

26. The method according to claim 25, wherein the aggregated data product is displayed on a display unit and the method comprises viewing the aggregated data product.

27. The method according to claim 25, wherein the method further comprises modifying an operational parameter of a medical device in response to receiving the aggregated data product.

28. The method according to claim 27, wherein the medical device is an implantable medical device.

29. The method according to claim 28, wherein the implantable medical device is a cardiac device.

30. An article, comprising:
a storage medium having instructions, that when executed by a computing platform, result in execution of a method of:
receiving continuous field tomography data;
receiving non-continuous field physiological data; and
producing an aggregated data product from the received continuous field tomography data and non-continuous field physiological data.
